(19) United States
(12) Patent Application Publication
(10) Pub. No.: US 2008/0249545 A1
(43) Pub. Date: Oct. 9, 2008

(54) SUTURING, CRIMPING AND CUTTING DEVICE

(75) Inventor: Oleg Shikhman, Trumbull, CT (US)

Correspondence Address: CANTOR COLBURN, LLP, 20 Church Street, 22nd Floor, Hartford, CT, 06103, US

(73) Assignee: INTERVENTIONAL THERAPIES, Westport, CT (US)

(21) Appl. No.: 12/098,665

(22) Filed: Apr. 7, 2008

Related U.S. Application Data
(60) Provisional application No. 60/922,344, filed on Apr. 6, 2007.

(51) Int. Cl. A61B 17/04

(52) U.S. Cl. 606/144

(57) ABSTRACT

A percutaneous surgical device is provided, which comprises a combination wound suturing and crimping and cutting device. In one exemplary embodiment a crimping and cutting device portion nests within a suturing device portion. The combined device may locate a vessel wound and pass suture through the vessel walls surrounding the wound. Then, the crimping and cutting portion may detach, the suturing portion may be removed, and the crimping and cutting portion may be located to the wound site to apply a fastener (e.g., a ferrule).

### CROSS-REFERENCE TO RELATED APPLICATIONS

The present application claims priority to U.S. Provisional Patent Application Ser. No. 60/922,344, filed Apr. 6, 2007 and to U.S. patent application Ser. No. 10/277,793, filed Oct. 22, 2002, which claimed priority to U.S. Provisional Application No. 60/345,212, filed Oct. 22, 2001, the entire disclosures of which are specifically incorporated by reference herein.

### BACKGROUND

When performing catheterization procedures, such an angiography or angioplasty, a catheter is generally introduced percutaneously (i.e., through the skin) into the vascular system by first penetrating the skin and underlying tissue, and then the blood vessel with a sharpened hollow needle. Location of a blood vessel, such as an artery, is typically achieved by feeling for the pulse, since such structures usually cannot be seen through the skin. Next, a guide wire is commonly inserted through the lumen of the hollow needle and is caused to enter the selected blood vessel. Subsequently, the needle is typically slid off the guide wire and a combination of a dilator and sheath are fed over the guide wire and pushed through the skin to enter the vessel. The guide wire and dilator can then be removed, and the desired catheter used to carry out the procedure is fed through the lumen of the sheath and advanced through the vascular system until the working end of the catheter is appropriately positioned. Following the conclusion of the catheterization procedure, the working catheter will be withdrawn and, subsequently, the sheath can also be removed from the wound, or left in place to facilitate closure.

At this point in the procedure, the vessel leakage is controlled in order to stem the flow of blood through the puncture. Because it is common practice to administer a blood thinning agent to the patient prior to many of the catheterization procedures, stemming the blood flow can be troublesome. A common method of sealing the wound is to maintain external pressure over the vessel until the puncture naturally seals. This method of puncture closure typically takes at least thirty minutes, with the length of time usually being substantially greater if the patient is hypertensive or anti-coagulated. In some anti-coagulated patients, the sheath is left in place for hours to allow the anti-coagulant to wear off. When human hand pressure is utilized, it can be uncomfortable for the patient and can use costly professional time on the part of the hospital staff. Other pressure techniques, such as pressure bandages, sandbags or clamps, have been employed, but these devices also require the patient to remain motionless for an extended period of time and the patient must be closely monitored to ensure their effectiveness.

There remains a need in the art for effective percutaneous tissue closure that is quick, easy to instruct and easy to learn, effective and comfortable for the patient.

### SUMMARY

The above described and other disadvantages of the prior art are overcome and alleviated by the present percutaneous surgical device, which comprises a combination wound suturing and crimping and cutting device.

In one exemplary embodiment a crimping and cutting device portion nests within a suturing device portion. The combined device may locate a vessel wound and pass suture through the vessel walls surrounding the wound. Then, the crimping and cutting portion may detach, the suturing portion may be removed, and the crimping and cutting portion may be located to the wound site to apply a fastener (e.g., a ferrule).

An exemplary wound suturing device comprises a housing and an elongated shaft connected thereto and at least one needle within the shaft, the at least one needle configured to travel distally across a tissue engaging gap within a tissue engaging section positioned distally from the housing on said shaft, wherein the tissue receiving gap has two opposing surfaces into which one side of a wound can be received, wherein the gap is shaped to have a depth to facilitate the placement of the edge of a wound therein such that at least one surface comprises a stop surface, wherein the stop surface is squared at a middle portion thereof to provide good tactile feel to the surgeon when tissue is engaged.

An exemplary crimping and cutting device comprises a hammer head having a first side and an opposite second side, and a ferrule engaging edge located on the second side, a tip having a distal end and a proximal end, the tip having a hammer head opening for receiving the hammer head, the hammer head opening extending from the distal end to the proximal end, the tip further having a ferrule accepting opening near the distal end, and a cutting edge within the hammer head opening of the tip, the cutting edge located proximally of the ferrule accepting opening.

The above described and other features are exemplified by the following figures and detailed description.

### BRIEF DESCRIPTION OF THE DRAWINGS

Referring now to the figures wherein the like elements are numbered alike:

FIG. 1A is a side plan view of the an exemplary wound suturing apparatus;

FIG. 2A is a bottom perspective view of an exemplary retainer member;

FIGS. 3A-5A illustrate perspective views of an exemplary actuation mechanism;

FIG. 6A is a perspective view of an exemplary slip-free mechanism;

FIGS. 7A and 8A are cross sectional and perspective views, respectively, of an exemplary tissue engagement section;

FIG. 9A is a cross sectional view of an exemplary suture and ferrules;

FIG. 10A is a cross sectional view of an exemplary needle and spherical member;

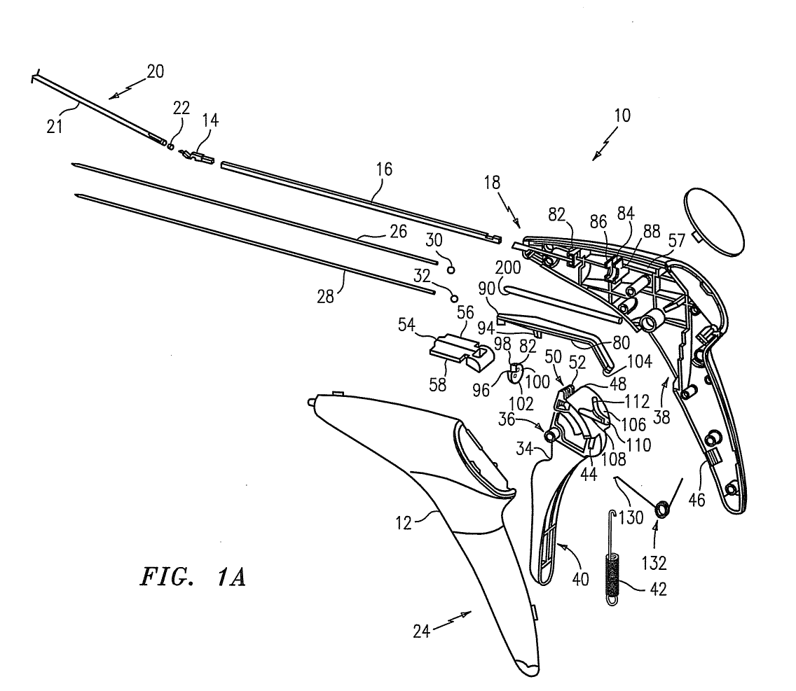

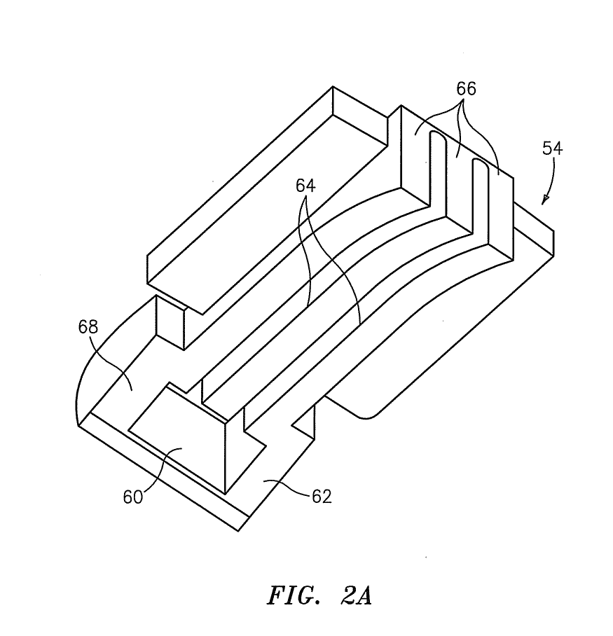

FIG. 1B shows an exploded perspective view of the cutting and crimping device;

FIG. 2B shows a side plan view of an exterior of the handle assembly for the cutting and crimping device of FIG. 1B;

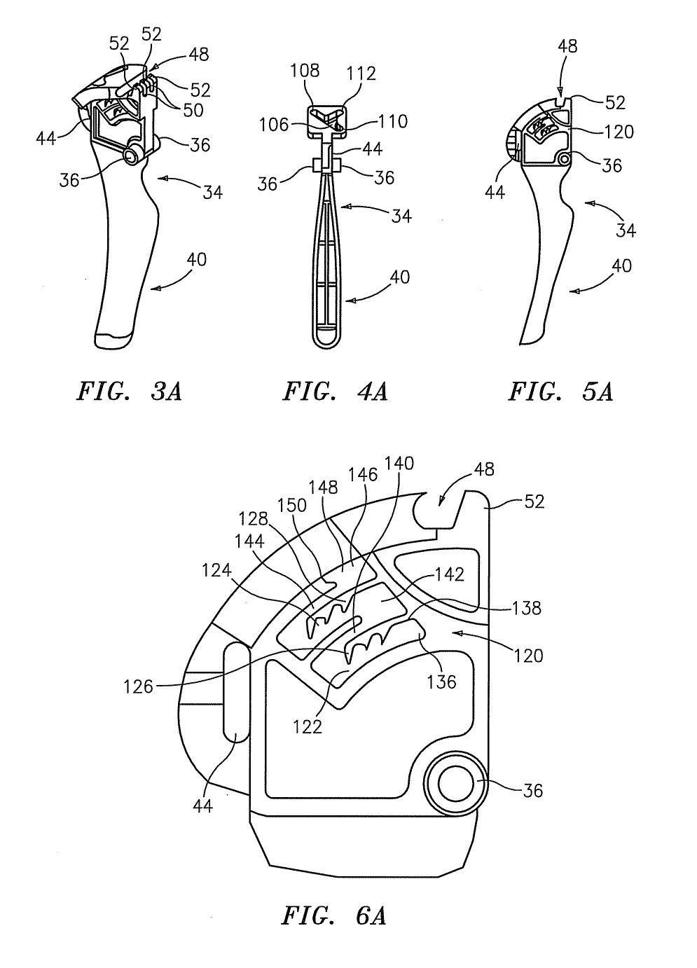

FIG. 3B shows an interior plan view of a side of the handle assembly for the cutting and crimping device of FIG. 1B;

FIG. 4B shows a proximal plan view of a side of the handle assembly for the cutting and crimping device of FIG. 1B;

FIG. 5B shows a proximal plan view of another side of the handle assembly for the cutting and crimping device of FIG. 1B;

FIG. 6B shows a partial cross-sectional view of the side of FIG. 4B;

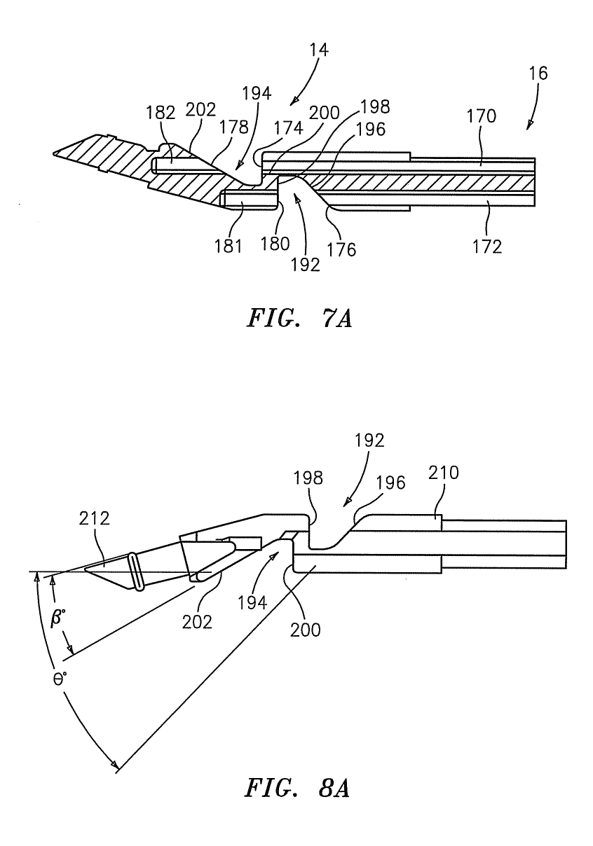

FIG. 7B shows a partial cross-sectional view of the side of FIG. 5B taken along line 7-7 of FIG. 3B;

FIG. 8B shows a side plan view of the trigger of the handle assembly for the cutting and crimping device of FIG. 1B;

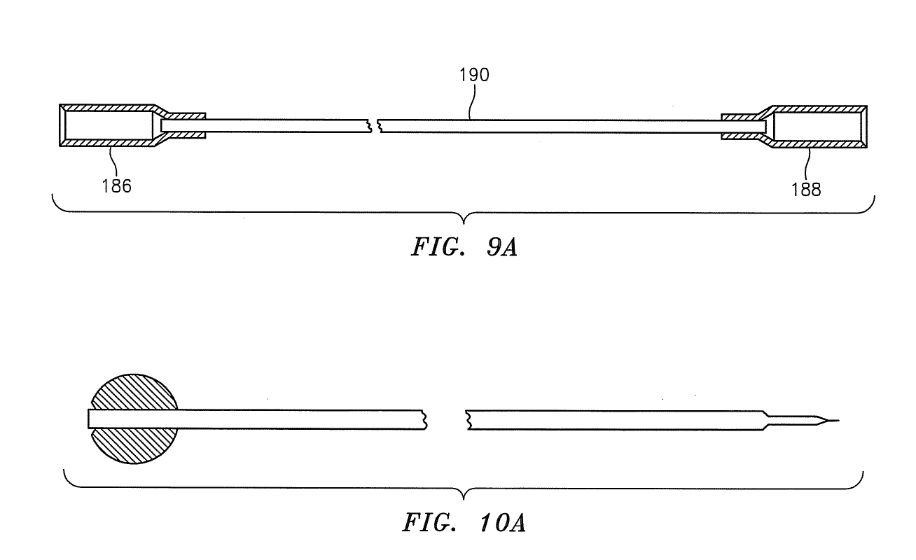

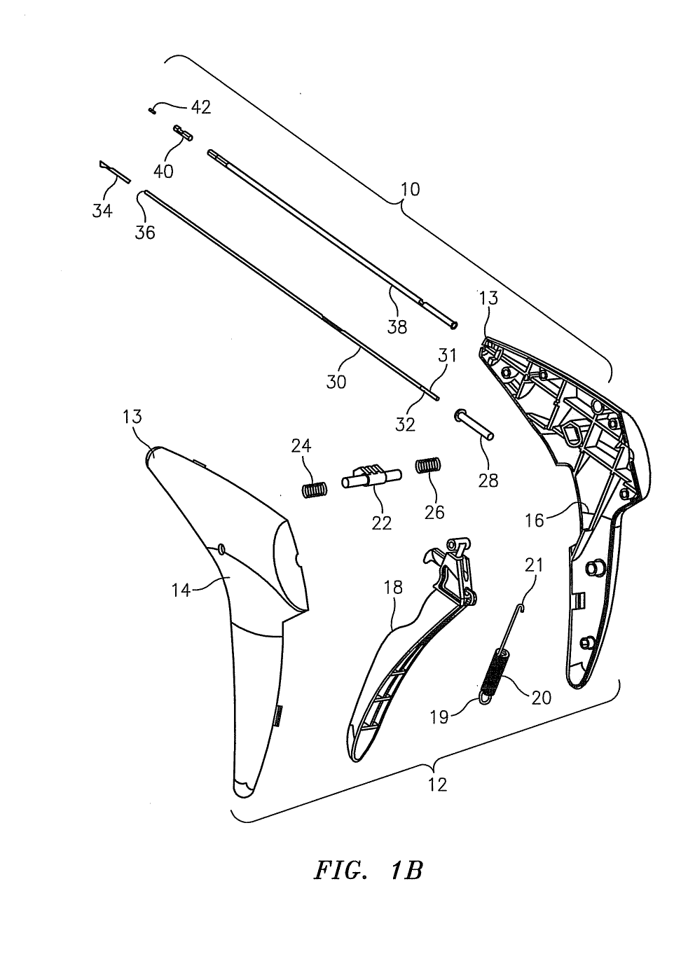

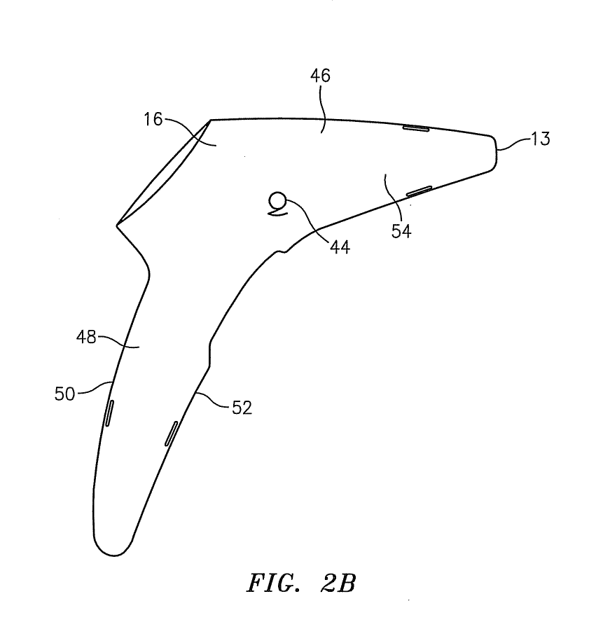

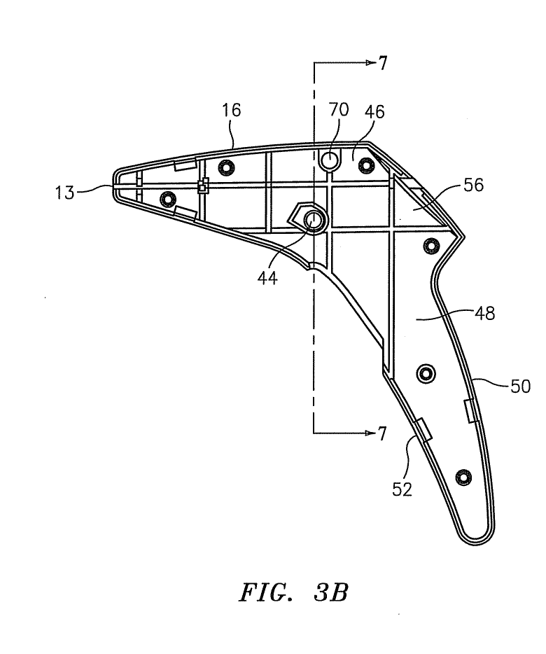

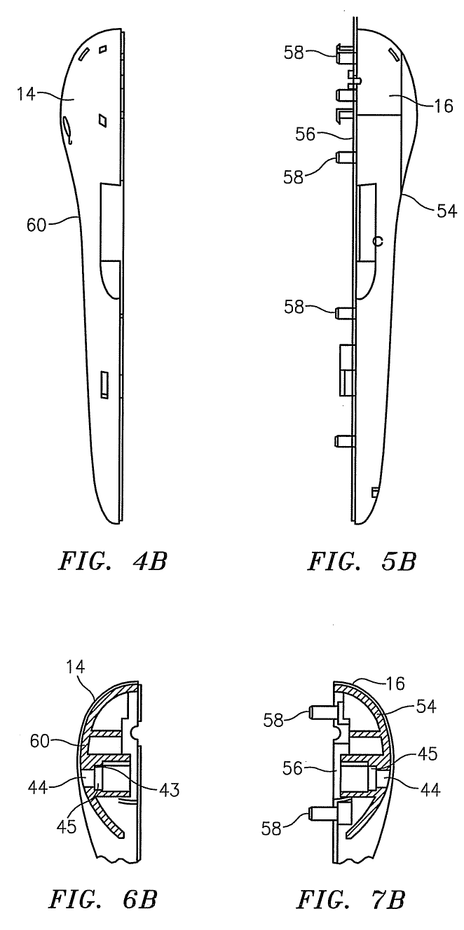

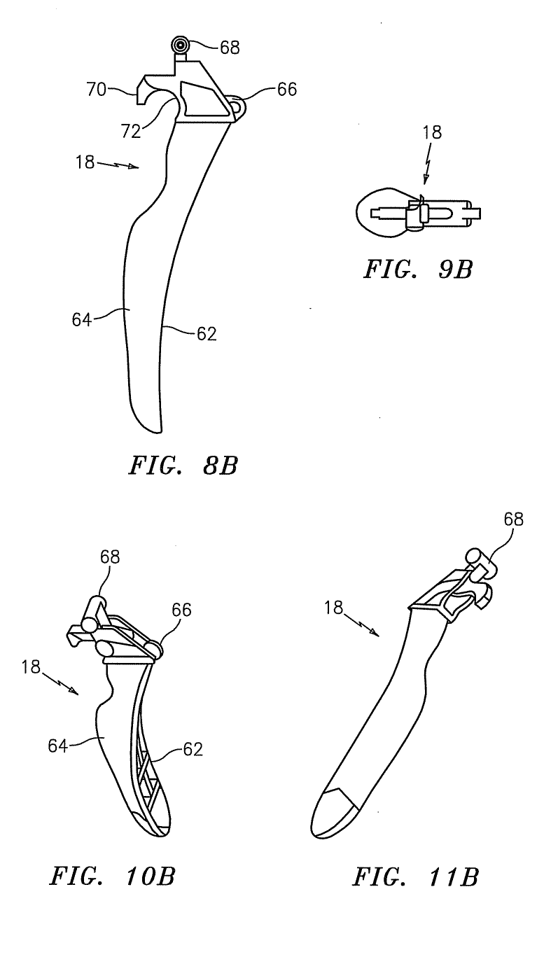

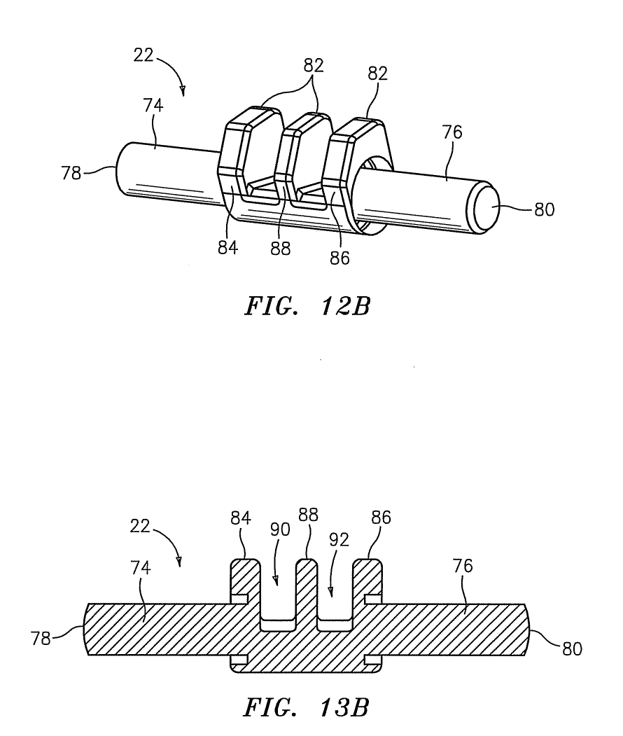

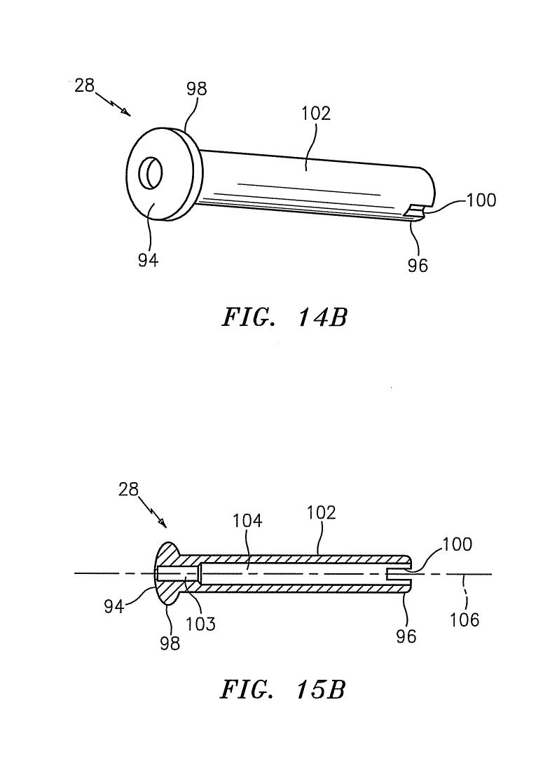

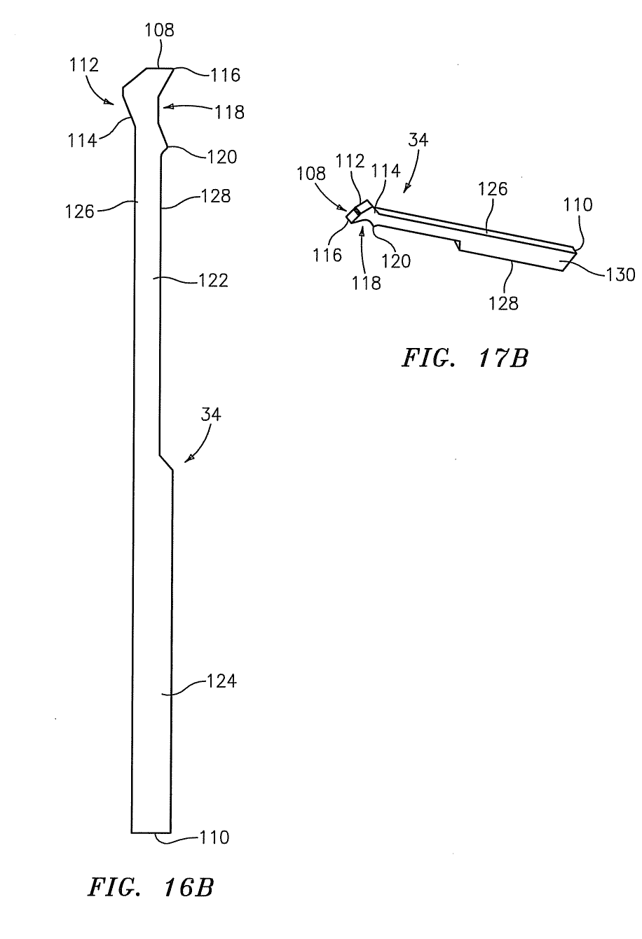

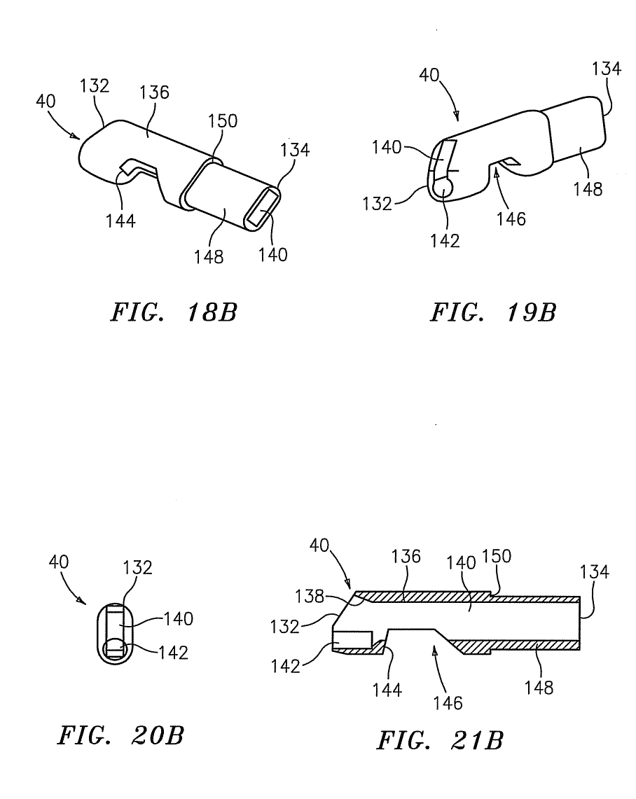

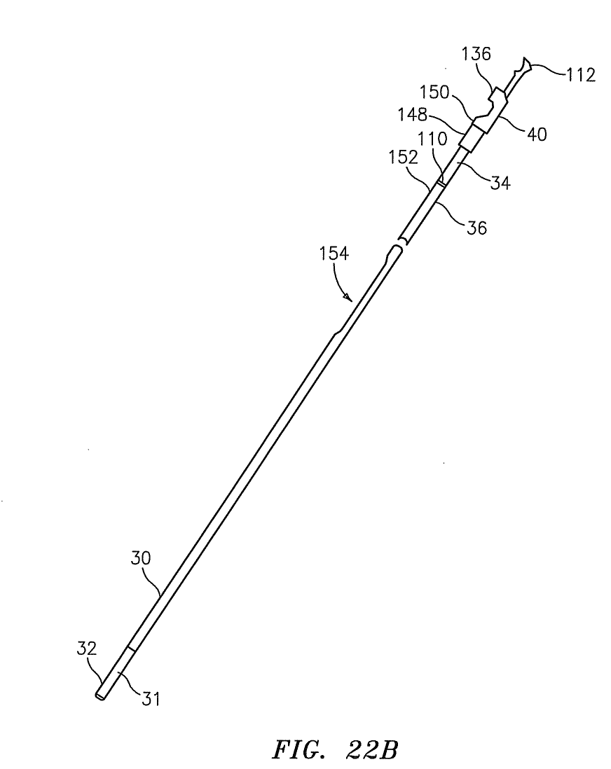

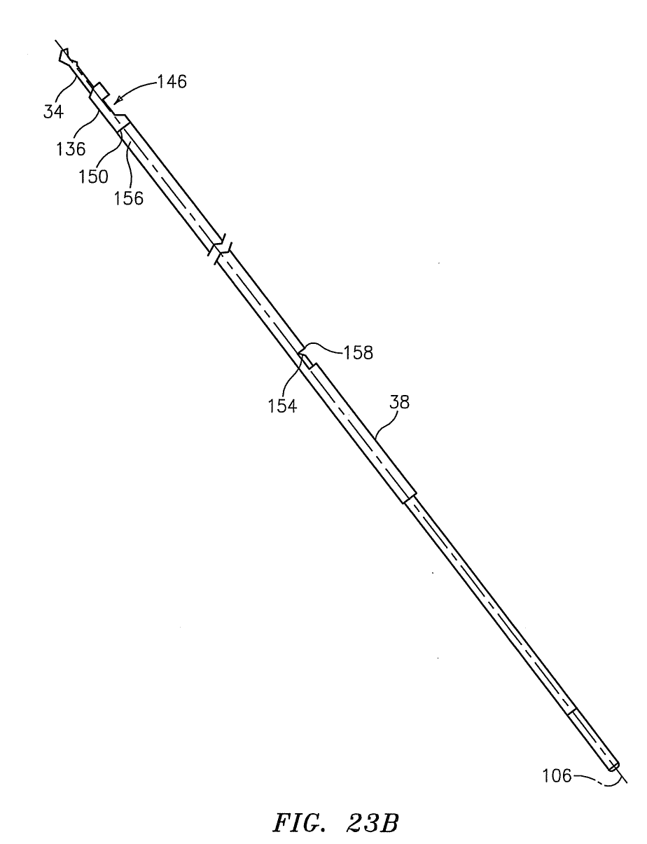

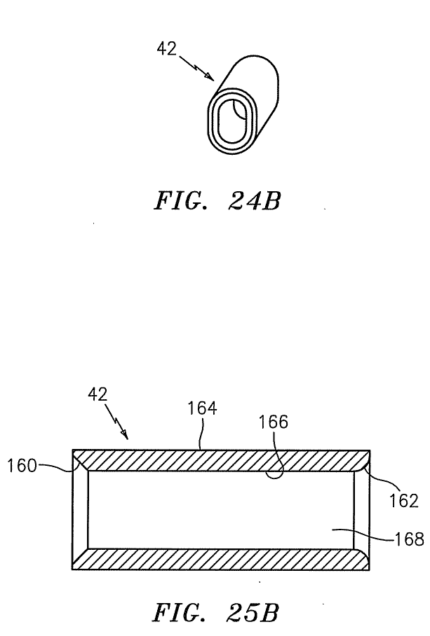

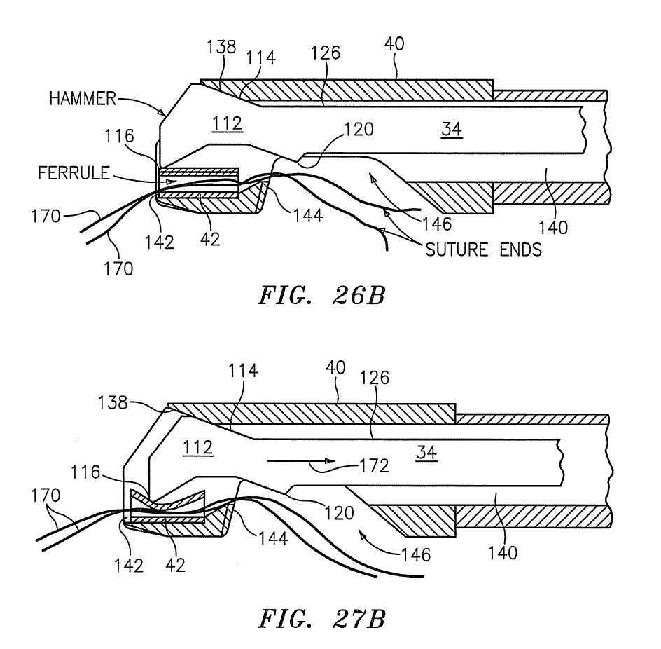

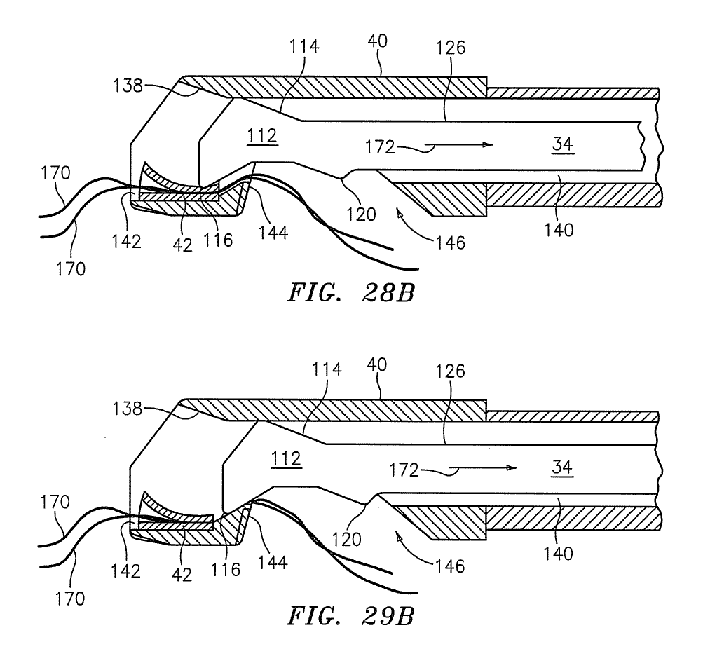

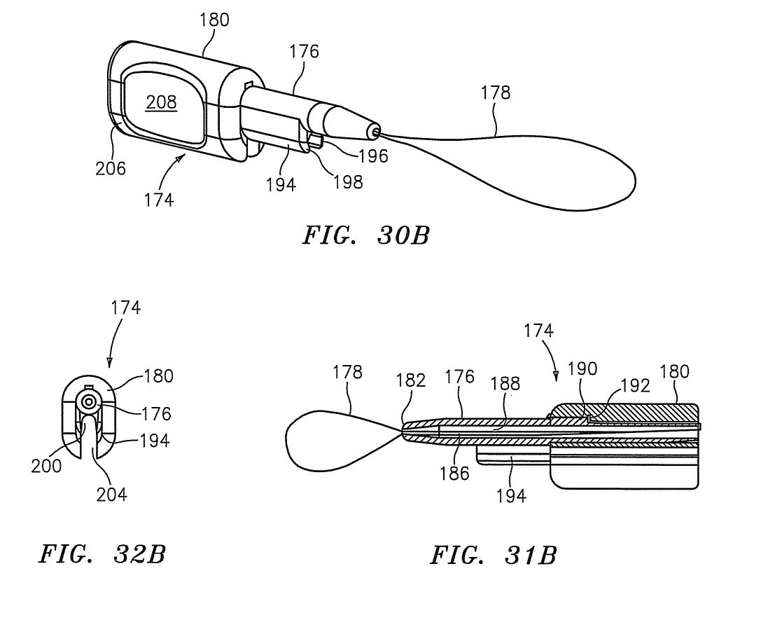

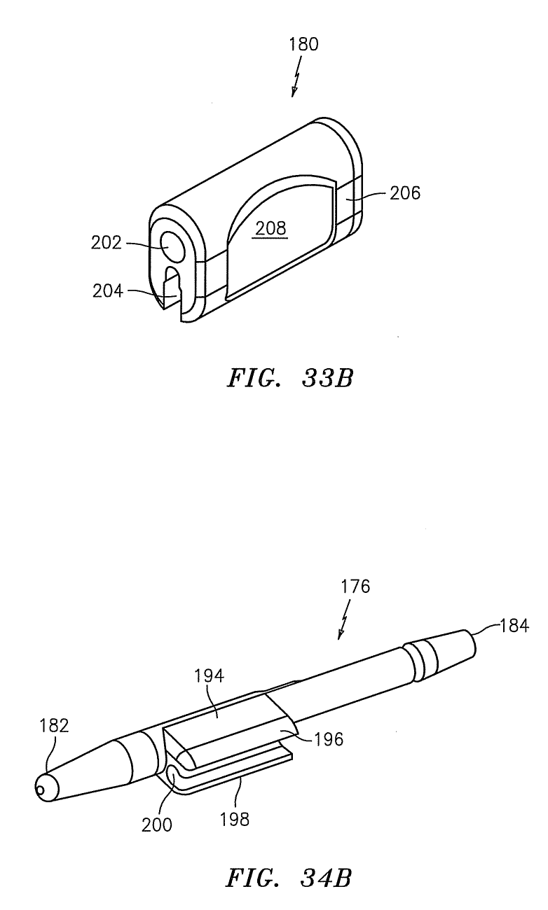

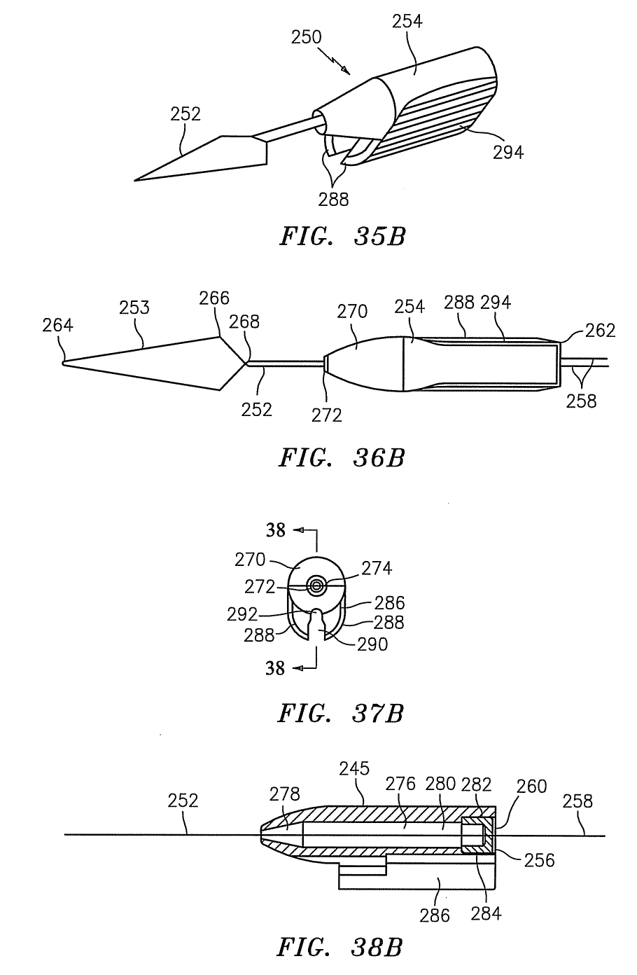

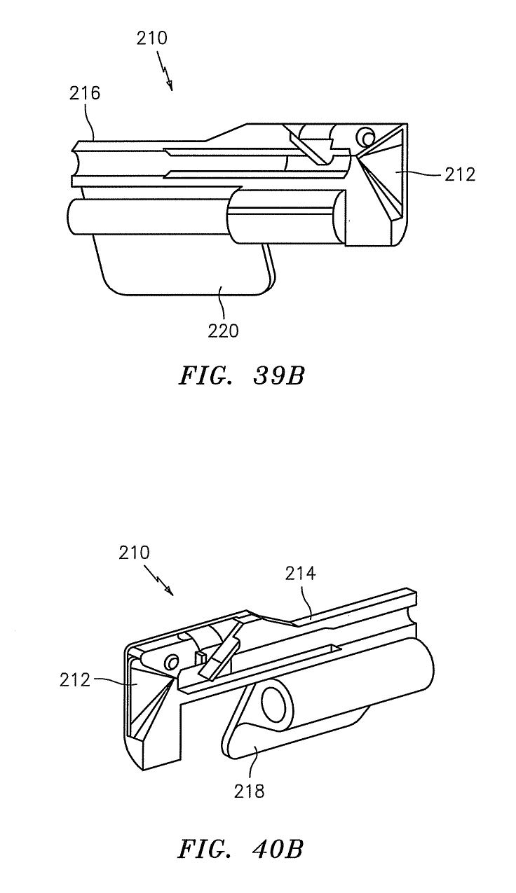

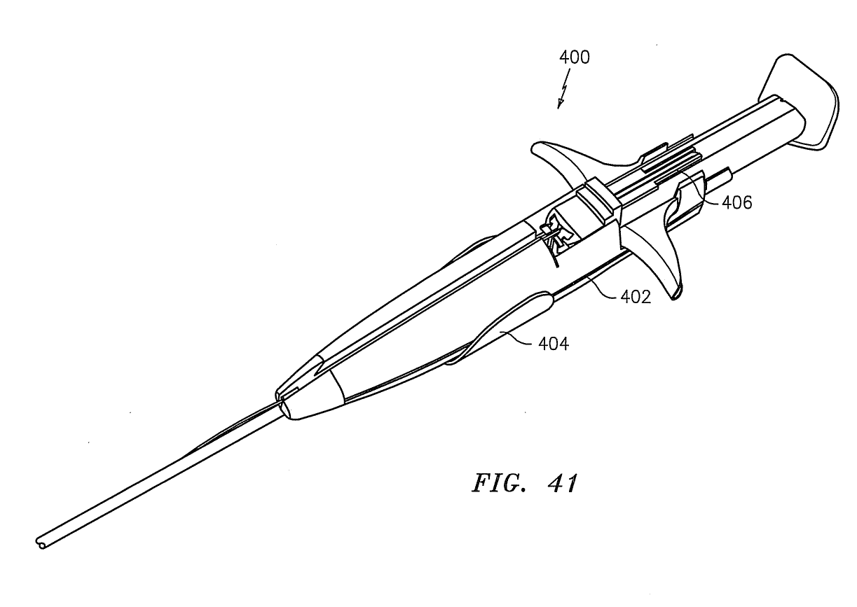

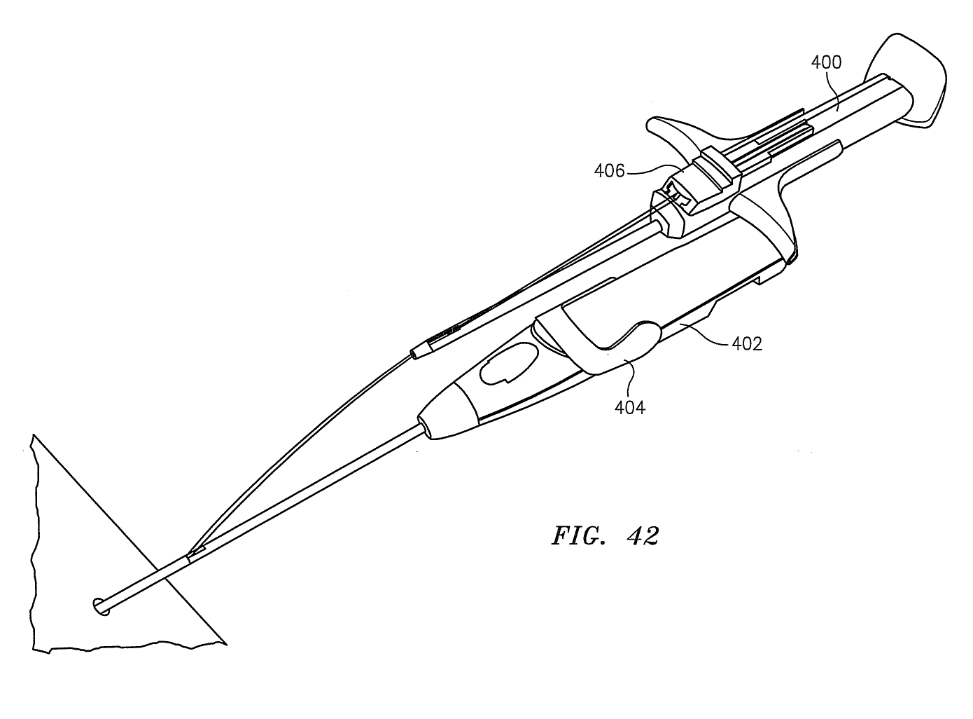

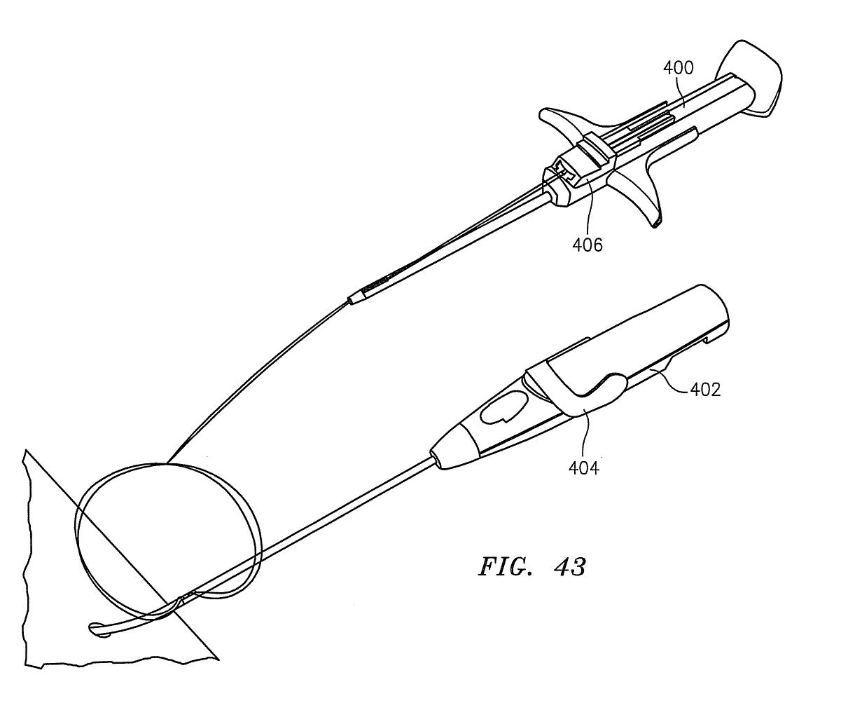

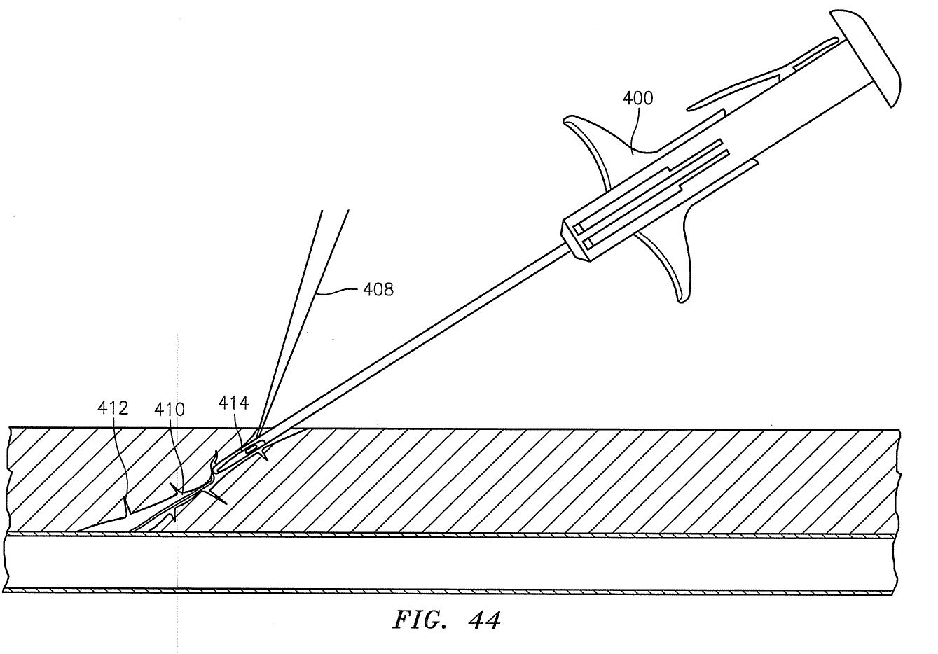

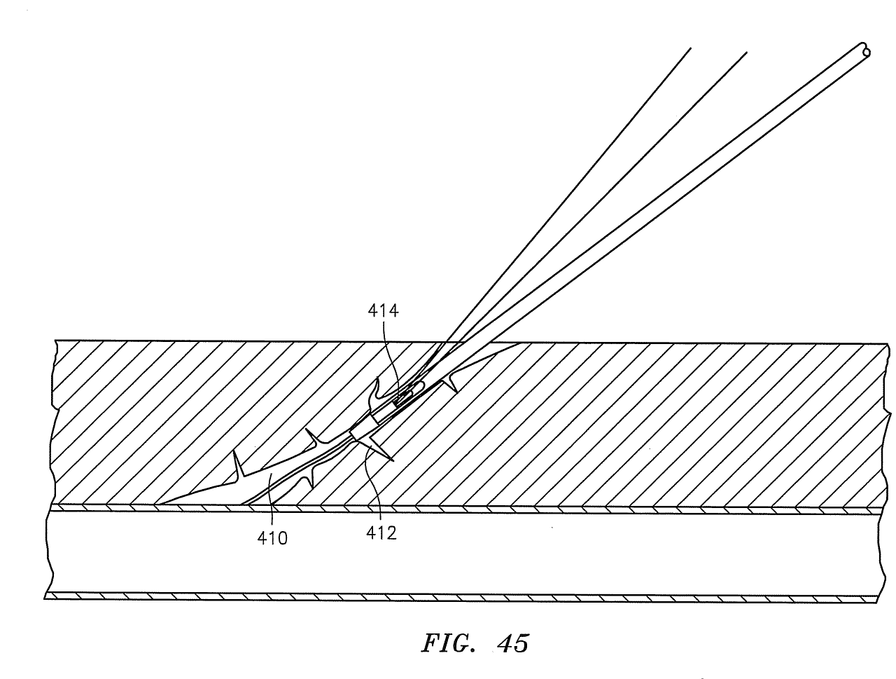

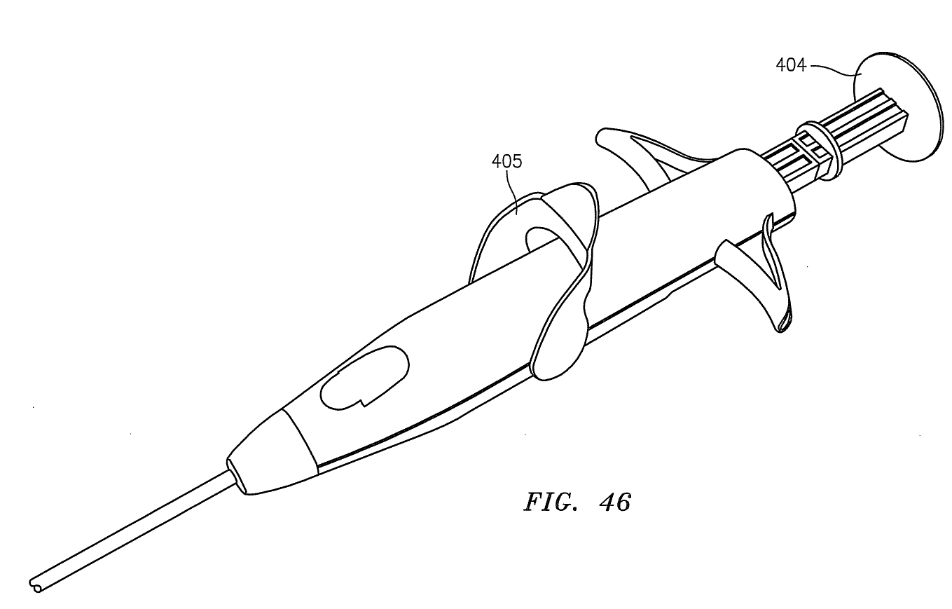

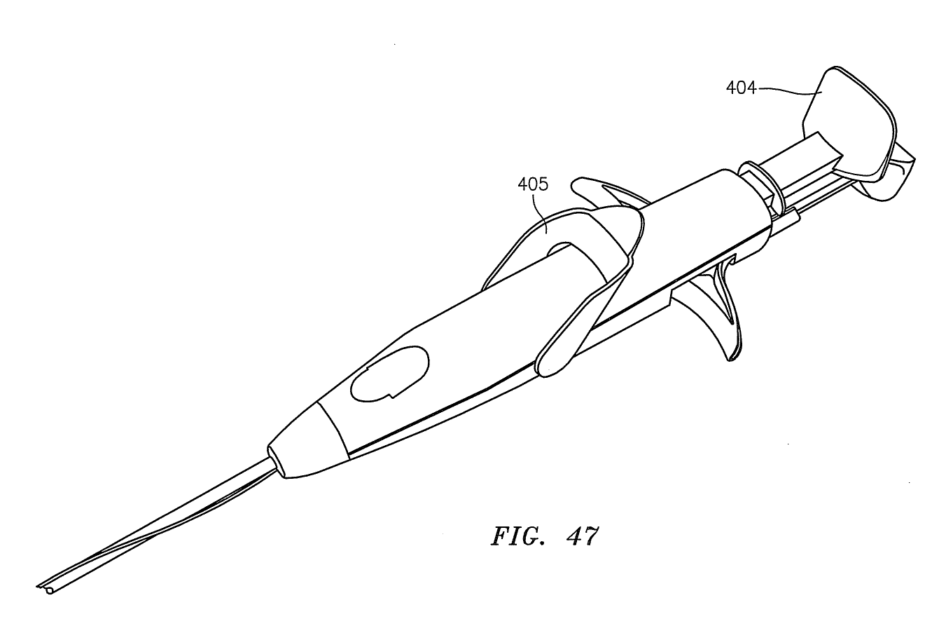

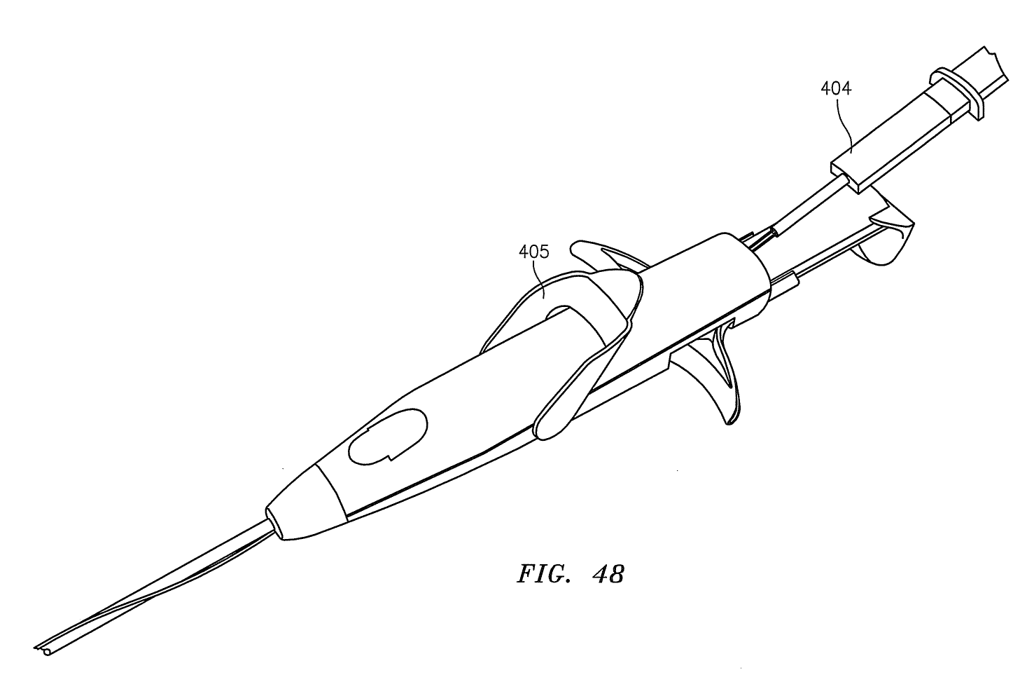

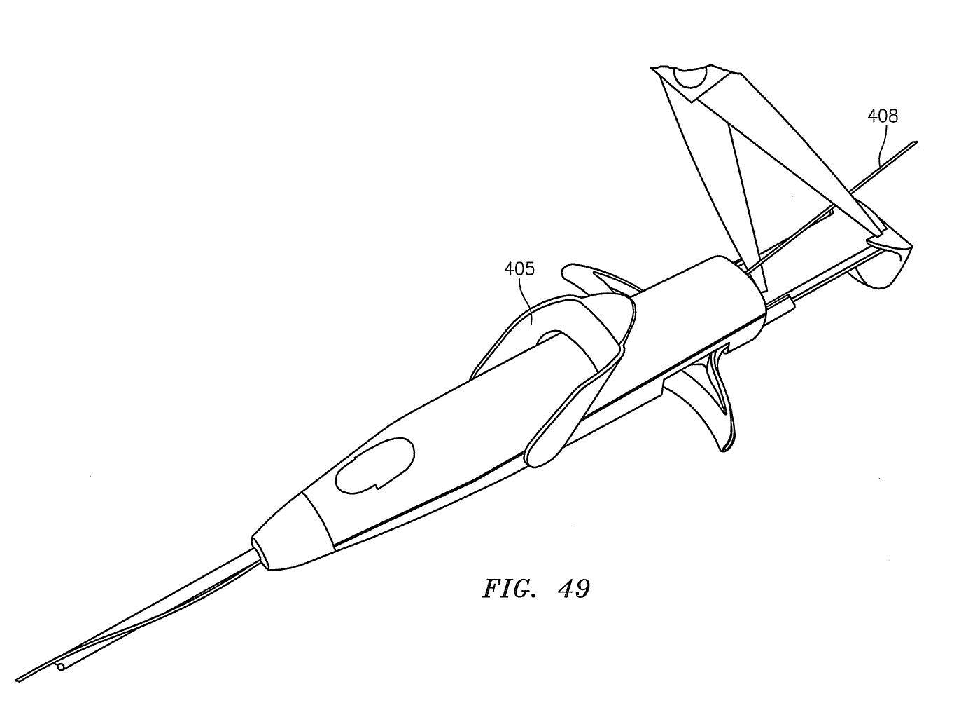

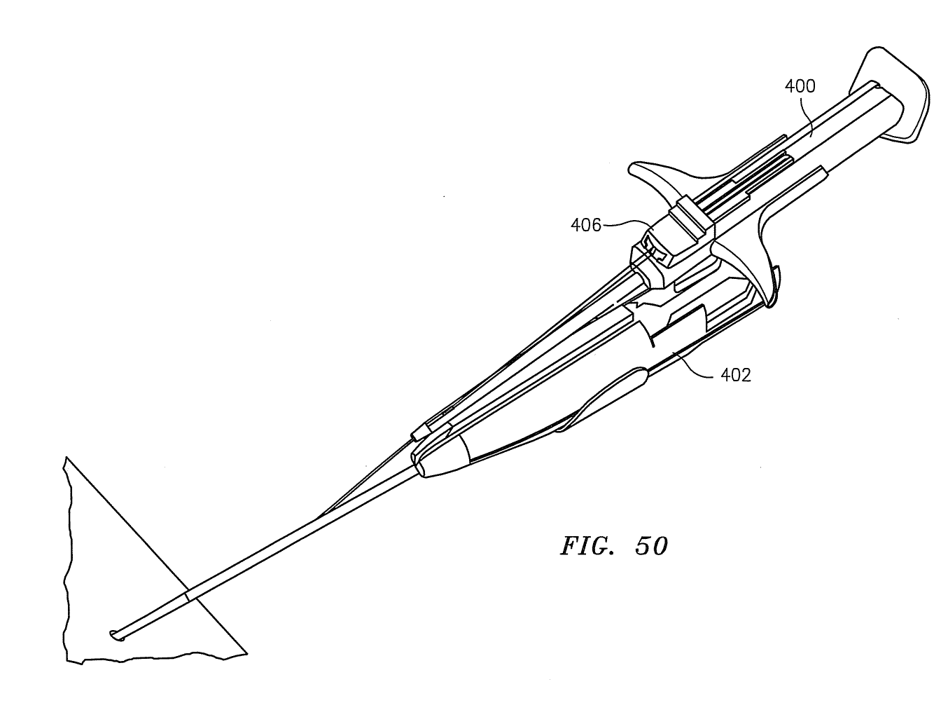

FIG. 9B shows a top plan view of the trigger of FIG. 8B;

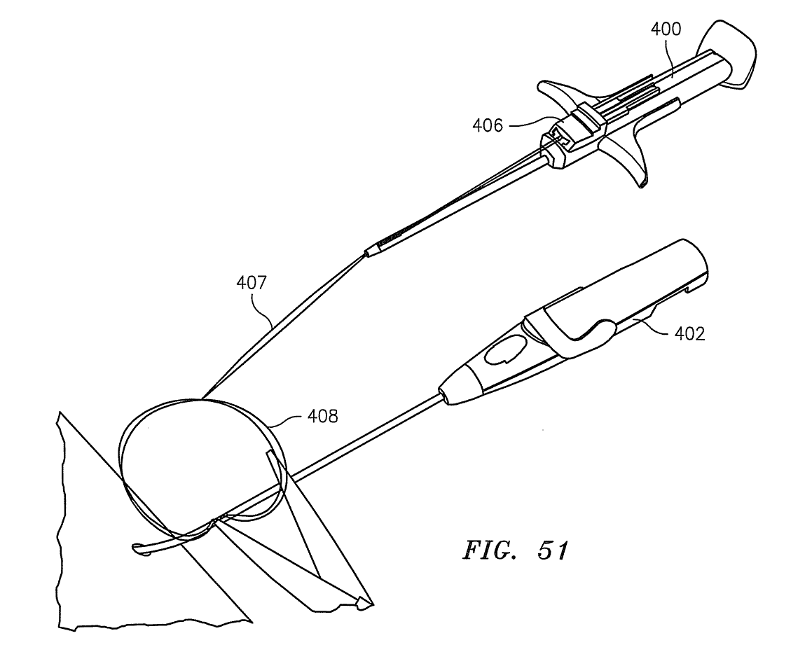

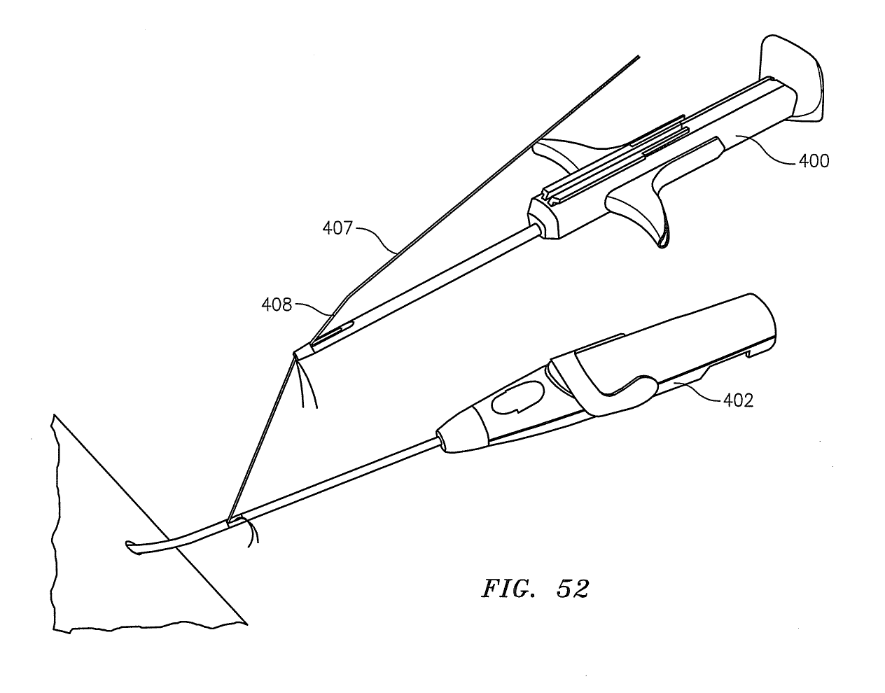

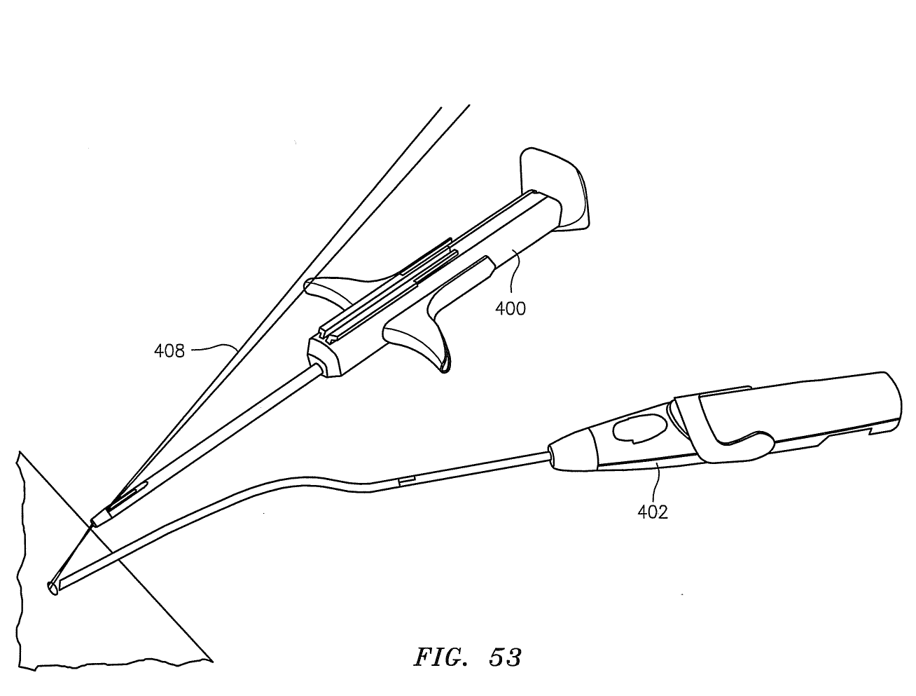

FIG. 10B shows proximal perspective view of the trigger of FIG. 8B;

FIG. 11B shows a distal perspective view of the trigger of FIG. 8B;

FIG. 12B shows a perspective view of the safety button of the handle assembly for the cutting and crimping device of FIG. 1B;

FIG. 13B shows a cross-sectional view of the safety button of FIG. 12B;

FIG. 14B shows a perspective view of the adjustment screw of the handle assembly for the cutting and crimping device of FIG. 1B;

FIG. 15B shows a cross-sectional view of the adjustment screw of FIG. 14B;

FIG. 16B shows a side plan view of the hammer element for the cutting and crimping device of FIG. 1B;

FIG. 17B shows a perspective view of the hammer element of FIG. 16B;

FIG. 18B shows a perspective view of the tip for the cutting and crimping device of FIG. 1B;

FIG. 19B shows another perspective view of the tip of FIG. 18B;

FIG. 20B shows a distal plan view of the tip of FIG. 18B;

FIG. 21B shows a cross-sectional interior view of the tip of FIG. 18B;

FIG. 22B shows a side plan view of the central rod, hammer element, and tip of the cutting and crimping device of FIG. 1B;

FIG. 23B shows a side plan view of the central rod, hammer element, tip, and tubular portion of the cutting and crimping device of FIG. 1B;

FIG. 24B shows a perspective view of a ferrule for use in the cutting and crimping device of FIG. 1B;

FIG. 25B shows a side cross-sectional view of the ferrule of FIG. 24B;

FIG. 26B shows a partial side cross-sectional view of the cutting and crimping device of FIG. 1B in an initial stage of securing suture material;

FIG. 27B shows a partial side cross-sectional view of the cutting and crimping device of FIG. 1B in an advanced stage of securing suture material;

FIG. 28B shows a partial side cross-sectional view of the cutting and crimping device of FIG. 1B in a further advanced stage of securing suture material;

FIG. 29B shows a partial side cross-sectional view of the cutting and crimping device of FIG. 1B in a final stage of securing suture material;

FIG. 30B shows a perspective view of one embodiment of a suture loading assembly;

FIG. 31B shows a side cross-sectional view of the suture loading assembly of FIG. 30B;

FIG. 32B shows a front plan view of the suture loading assembly of FIG. 30B;

FIG. 33B shows a side perspective view of a cap for use in the suture loading assembly of FIG. 30B;

FIG. 34B shows a side perspective view of a body for use in the suture loading assembly of FIG. 30B;

FIG. 35B shows a side perspective view of another embodiment of a suture loading assembly;

FIG. 36B shows a top plan view of the suture loading assembly of FIG. 35B;

FIG. 37B shows a front plan view of the suture loading assembly of FIG. 35B;

FIG. 38B shows a side cross-sectional view of the suture loading assembly of FIG. 35B taken along line 38-38 within FIG. 37B;

FIG. 39B shows a side perspective view of a half of another embodiment of a suture loading assembly; and,

FIG. 40B shows a side perspective view of another half of the suture loading assembly of FIG. 39B;

FIG. 41 is a bottom perspective view of a combination suturing device, crimper and cutter;

FIG. 42 is a top perspective view of a cutting and crimping portion partially disassociated from a suturing device; and

FIG. 43 is a perspective view of a cutting and crimping portion disassociated from a suturing device but connected to suture.

FIGS. 44-53 are sequential illustrations of the workings of the combined device.

### DETAILED DESCRIPTION

The above described and other disadvantages of the prior art are overcome and alleviated by the present wound suturing device, which comprises a combination wound suturing and crimping and cutting device.

In one exemplary embodiment a crimping and cutting device portion 400 nests within a suturing device portion 402. The combined device may locate a vessel wound and pass suture through the vessel walls surrounding the wound (suture placement may be actuated or facilitated by one or more levers 404.

Referring to FIG. 46, the device may also include an extension actuating lever 405, which extends or rotates a part of the device at the operative section (e.g., extending a foot portion to create a tissue receiving gap through which the needle will extend upon actuating the suturing needles via lever 404, shown in FIG. 47).

FIG. 48 shows withdrawal of the suture needle actuating lever 404, which pulls the captured suture thread through the body of the suturing device 402. FIG. 49 shows cutting of the suture thread 408 after withdrawal of lever 404.

Then, the crimping and cutting portion 400 may detach (as in FIGS. 42 and 50), the suturing portion 402 may be removed, and the crimping and cutting portion 400 may be located to the wound site to apply a fastener (e.g., a ferrule).

The crimping and cutting portion 400 may also include a suture loading device 406 that facilitates threading of a suture through the ferrule. With specific regard to FIG. 51, detachment of the crimping and cutting portion 400 may also pull a length of suture from the body of the suturing portion 402. The suture may be cut proximally of the suture loading cable 407, and the suture loading device may be retracted proximally to pull the suture through the tip of the crimping and cutting portion 400 (as shown in FIG. 52).

The crimping and cutting portion 400 may then be advanced to the wound utilizing the suturing portion 402 as a guide.

This combination design reduces problems with repeatability of locating the fastener on the suture directly above the wound. An improper angle of insertion through the skin and down the tract of the wound can result in serrated or intermediate tissue being trapped between the ferrule and the blood vessel wound. By using the original suturing device/locator as a reference, more consistent fastener placement is achieved. FIGS. 44-53 exemplify this.

Specifically, FIG. 43 illustrates detachment of the crimping and cutting portion 400 from the suturing portion 402 after the wound has been sutured. The suture 408 is then loaded through the ferrule (housed in a distal tip portion of the crimping and cutting portion 400, not shown in FIG. 43) by proximal sliding of the suture loading device 406.

FIGS. 44 and 45 illustrate how the tissue tract 410 is not smooth, but rather includes fissures 412 that can catch the tip portion 414 of the crimping and cutting portion 400 if the angle of insertion is slightly off. By providing the crimping and cutting portion and the suturing portion as a combined device, the suturing portion may be used as a reference when inserting the crimping and cutting portion, resulting in more consistent fastener placement.

### Descriptions of Exemplary Suturing Devices

An apparatus for applying a suture to body tissue is illustrated in FIG. 1A and is designated generally by reference numeral 10. Note that the terms “first” and “second” as used herein are for the reader's convenience and should not be interpreted as necessarily denoting the order in which the components are actuated.

Referring to FIG. 1A, an exemplary wound suturing apparatus 10 is shown having a housing 12, a tissue engaging portion 14, a shaft 16 extending from an opening 18 in the housing to the tissue engaging section 14, and a flexible guide tube 20 coupled at 22 to the tissue engaging section 14. The housing 12 has a body shaped like a pistol having a handle portion 24, and is illustrated in the exemplary embodiment as a two-piece construction of molded plastic. The apparatus 10 includes a pair of needles 26 and 28, which extend from housing 12 through the shaft 16 into the tissue engaging section 14. Each needle 26 and 28 has a non-tissue engaging end in the housing having a spherical member 30 and 32, such as a ball or bearing, respectively, attached thereto. Both needles 26 and 28 and spherical members 30 and 32 may be a made of metal, such as surgical stainless steel. The spherical members 30 and 32 may have a bore into which the non-tissue engaging ends of the needles 26 and 28, respectively, extend and joined thereto, such as by welding.

The apparatus 10 includes an actuator member 34 having two pins 36 extending into holes in the sides of housing 12 upon which the actuator member is pivotally mounted in the housing. Actuator member 34 has a portion that extends through an opening 38 in housing 12 to provide a trigger 40. A coil spring 42 is provided which hooks at one end in a notch 44 of actuator member 34 and is wound at the other end around a pin 46 located in holes in the sides of housing 12, such that the actuator member 34 is spring biased to retain trigger 40 normally in a forward position, as shown for example in FIG. 1A. A notch 48 is provided in the actuator member 34 which is shaped to receive one of the non-engaging ends of needles 26 or 28, i.e., spherical members 30 or 32, to be driven forward by the actuator member 34 by a user pulling the trigger portion 40 of actuator member 34 towards handle portion 24. Two grooves 50 are provided by three fingers 52 into which portions of the needles 26 or 28 proximate to the spherical members 30 or 32, respectively, may lie.

A retainer member 54 is fixed in housing 12 by two flanges 56, 58 above the actuator member 34 within mating surfaces 57. As best shown in FIG. 2A, the retainer member 54 has a chamber 60 having a lower opening 62 and two grooves 64 formed by fingers 66 which allow the spherical members 30 or 32 of needles 26 or 28, respectively, to be received in chamber 60 to restrict movement of the needle when held therein. The lower surface 68 of retainer member 54 is curved and faces correspondingly shaped fingers 52 of actuator member 34, such that the actuator member 34 is slidable along lower surface 68 responsive to a user pulling and releasing trigger 34.

Referring now to FIGS. 3A-6A, an exemplary actuator mechanism 34 is shown in detail by the various perspective views. As described above, the actuator mechanism 34 includes a trigger portion 40, pins 36, a notch 44 for receipt of a coil spring 42, a notch 48 shaped to receive one of the non-engaging ends of needles 26 or 28, and grooves 50 provided by three fingers 52 into which portions of the needles 26 or 28 proximate to the spherical members 30 or 32, respectively, may lie.

Referring again to FIG. 1A, a needle selection mechanism is provided including a selector lever (or arm) 80, which is rotationally coupled with a cam member 82. The cam member 30 is supported by an adapter 84 in housing 12. The cam member 82 is mounted in housing 12 by two flanges 86, 88. The selector lever 80 is pivotally mounted by a pin 90 extending downwards from a distal end portion of the selector lever into a notch 92 in the housing 12. The selector lever 80 has a downwardly protruding member 94 which is received in a notch 96 of cam member 82 to rotate cam member 82 in a pocket between flanges 86 and 88 as the selector lever 90 is moved left or right. The cam member 82 has a tapered surface 98 to facilitate its rotation in pocket and two tapered apertures 100 and 102 through which needles 26 and 28 respectively extend. The selector lever 80 further includes a proximal pin 104 configured to engage a slot, shown generally at 106 on an upper portion of the actuator member 34.

In an initial configuration, the proximal pin 104 is positioned in a left lobe 108 of slot 106. During an initial actuation of the trigger portion 40, the proximal pin 104 of the selector mechanism 80 travels to trough 110 of the slot 106. During this actuation, the orientation of the cam member 82 is such that the needle 28 is in an engaged position within the grooves 50, 48 of the actuator member 34 while the needle 26 is disengaged from the grooves 50, 48. The lobes 108, 112 and trough 110 of the slot 106 is configured such that release of the actuated trigger portion 40 causes the proximal pin 104 to travel into the right lobe 112 of the slot 106. This causes the proximal portion of the selector mechanism 80 to shift to the right, and at the same time, causes the cam member 82 to rotate in the same direction. The cam member 82 urges the needle 28 out of the grooves 50, 48 in the actuator mechanism 34 and simultaneously urges the needle 26 into the grooves 50, 48 in the actuator mechanism 34. Thus, a second actuation of the trigger portion 40 urges needle 26 in a distal direction but does not actuate the needle 28.

Referring now to FIGS. 5A and 6A, the actuator mechanism 34 is provided with a slip-free mechanism, shown generally at 120. The slip-free mechanism 120 generally comprises dual ratchet tracks 122, 124, each with a plurality of ratchet teeth 126, 128. The ratchet tracks and teeth are configured to engage a traveling pin portion 130 of spring 132 (shown in FIG. 1A). Referring to FIG. 6A, the traveling pin portion 130 resides initially at a distal region 136 of ratchet track 122 and is biased against an upper wall 138 of the ratchet track 122. During initial actuation of the trigger portion 40 of the actuation member 34, the traveling pin portion 130 traverses the ratchet teeth 126. After a full actuation of the trigger portion 40, the traveling pin portion 130 moves into a first return channel 140. Release of the trigger portion 40 positions the traveling pin portion 130 at a distal region 142 of the second ratchet track 124. During a second actuation of the trigger portion 40, the traveling pin portion 130 traverses the ratchet teeth 128. After a full second actuation of the trigger portion, the traveling pin portion 130 moves into a second return channel 144. Release of the trigger portion 40 positions the traveling pin portion 130 at a distal region 146 of a lockout track 148. The traveling pin portion 130 is thereafter prevented from further movement by lockout stop 150.

Thus, the slip-free mechanism 120 and the traveling pin portion 132 of the spring 130 allow for only two actuations of the actuator mechanism 34. At the same time, the ratchet teeth 126 and 128 within the ratchet paths 122 and 124 prevent partial actuation of the actuator mechanism 34 and thus, partial deployment of the needles 26, 28.

Referring to FIG. 7A, to orient the needles 26 and 28 within the tissue engagement section 14, the two needles 26 and 28 are configured to travel through generally parallel tracks 170, 172 in an x-z plane as they exit the shaft 16 and cross the tissue engaging section 14. To maximize the separation of the needles 26 and 28, the shaft 16 is oval in cross-section, having a major axis of the oval (though the cross-section may be circular or any convenient shape) for at least a substantial portion of the shaft as it extends to shaft end near the tissue engaging section 14. The tissue engaging section 14 of the tissue suturing apparatus 12 further includes a first opening 174, a second opening 176, and third 178 and fourth 180 openings providing access to distal channels 182 and 184, which are each capable of holding a needle capturing portion 186 and 188, respectively (see FIG. 9A), received through openings 178 and 180, respectively. Needle capturing portions 58a and 60a are referred to herein as ferrules, such as described, for example, in U.S. Pat. Nos. 5,431,666 and 5,766,183, but may be any means by which a suture may be captured at the tip of a needle. The ferrules 186 and 188 each have an opening to an interior cavity shaped to enable the ferrule to frictionally engage the end of the needles 26 and 28, respectively, when received in the interior cavity. Each ferrule may be made of metal or plastic and may be oval in cross-section such that they can frictionally engage the tip of a needle. The ferrules 186 and 188 are each connected to one end of the two ends of a length of suture material or thread 190 extending through a suture tube or channel (not shown) positioned either in the elongated body 16 or in the flexible member 20.

In another embodiment one or more of the ferrules 186, 188 includes an interior cavity with an angled cross section providing multiple lines of interference. In another embodiment, the interior cavities of the ferrules 186, 188 have triangular cross sections, providing three lines of interference during engagement of the ferrule interiors with the distal tips of the needles. In another embodiment, the interior cavities of the ferrules 186, 188 have square cross-sections, providing four lines of interference during engagement of the ferrule interiors with the distal tips of the needles.

With reference to FIGS. 7A and 8A, the tissue engaging section 14 has a first gap 192 and a second gap 194 in which the first gap 192 is along the lower side of section 14 and the second gap 194 is along the opposite upper side of section 14 and forward with respect to the first gap along the length of the section 14 in a direction distal from housing 12. The first gap 192 has two opposing surfaces 196 and 198 into which one side of a wound can be received, where opening 176 is located along surface 196 and opening 180 to ferrule holder 184 is located along surface 198 facing opening 176. Similarly, the second gap 194 has two opposing surfaces 200 and 202 into which the other side of the wound can be received, where opening 174 is located along surface 200 and the opening 178 to ferrule holder 182 is located along surface 202 and faces opening 174. Each gap 192 and 194 is shaped to have a depth to facilitate the placement of the edge of a wound therein. Surface 198, which is the distal face of the first gap 192, and surface 202, which is the proximal face of the second gap 194 both serve as stop surfaces for the tissue engaging section 14. Such stop surfaces 198, 200 assist in the placement of the tissue engaging section 14 relative to the wound as will be described further below. End portions 210 and 212 of the tissue engaging section are angled with respect to each other as shown in FIG. 8A to facilitate placement of end 212 with guide section 20 through a sheath (or cannula) and the puncture wound to maximize blood vessel engagement. The two ferrules 186 and 188 and suture material 190 may be located in apparatus 10 during manufacture.

With reference to FIG. 8A, in one embodiment, stop surfaces 198 and 200 are squared at a middle portion thereof to provide good tactile feel to the surgeon of the stop points. In another embodiment, the stop surfaces may have an angle with regard to the longitudinal axis of the first end portion 210 of the tissue engaging section 14 of between about 85 and 95 degrees. In another embodiment, the stop surfaces have an angle of about 90 degrees.

Referring still to FIG. 8A, in one embodiment, wall 196 of the first gap 192 has an angle theta of between about 40 and 50 degrees with regard to the longitudinal axis of the first end portion 210 of the tissue engaging section 14. In another embodiment, wall 196 has an angle theta of about 45 degrees.

Referring still to FIG. 8A, in one embodiment, wall 202 of the first gap 194 has an angle beta of between about 25 and 35 degrees with regard to the longitudinal axis of the first end portion 210 of the tissue engaging section 14. In another embodiment, wall 202 has an angle beta of about 30 degrees.

Thus, by the above described exemplary ranges of the geometry of the tissue engaging section 14, an aggressive tissue contacting surface is described to facilitate bite of tissue, particularly for wound suturing devices having small sizes, where positive tissue capture and stop indication is particularly advantageous. In one embodiment, the tissue engaging section 14 has a size between about 6 and 8 French. In another embodiment, the tissue engaging section 14 has a size of about 7 French.

The tissue engagement section 14 may be made of metal, such as stainless steel, or other rigid biocompatible material. For example, the tissue engagement section may be made of two pieces of shaped metal having bores providing the desired openings, channels, and receptacles, joined together down the middle along section by welding or heat shrinking of heat shrinkable tubing connecting the two pieces. The components in the housing 12, such as the actuator member 34, selector lever 80, and needle retainer 220, may be made of molded plastic.

A guide section 20 is attached to end 212 (FIG. 8A) of the tissue engaging section 14. As shown best in FIG. 1A, the guide section 20 has a flexible tube 21 having an opening (not shown) through which a guide wire may be received. The tube 20 may be made of a biocompatible plastic, like heat shrink tubing, and the ramp may be made of plastic or metal, which is attached or joined within tube 20.

### Descriptions of Exemplary Cutting and Crimping Devices

Referring to FIG. 1B, a crimping and cutting device 10 is shown for applying a ferrule around suture material after it has been applied to body tissue by a suturing apparatus, or by any other suturing procedure in which suture material is used, in order to secure the suture.

In general, device 10 preferably includes a handle assembly 12. The handle assembly may include first and second sides 14, 16 and a trigger member 18 with an associated spring 20 for the trigger return. The handle assembly 12 further preferably includes a safety button 22 which is centrally biased by springs 24, 26 and which must be depressed before trigger actuation will be permitted. Also preferably within the handle assembly 12 is an adjustment screw 28 which facilitates ferrule loading by the manufacturer. The adjustment screw is connected to a proximal end 32 of a central rod 30, which extends from a distal end 13 of the handle assembly 12. A hammer element 34 is connected to the distal end 36 of the central rod 30. Surrounding the central rod 30 is a tubular portion 38 which also extends from a distal end 13 of the handle assembly 12. A tip 40 is secured to the proximal end of the tubular portion 38. Positioned within the tip 40 is a ferrule 42.

An exterior 54 of side 16 of handle assembly 12 is shown in FIG. 2B and FIG. 3B shows an interior 56 of side 16, similar to that shown in FIG. 1B. The handle assembly 12 includes a distal end 13 from which the central rod 30 and tubular portion 38 extend. The side 16 includes an opening 44 through which a portion of the safety button 22 extends, as will be further described. The side 16, as with the side 14, preferably includes a body portion 46 and a grasping portion 48. The body portion 46 houses the safety button 22 and the adjustment screw 28 and includes the connections for the central rod 30, tubular portion 38, and trigger 18. The grasping portion 48, on the hand, preferably includes an ergonomically shaped grip for an operator. The grasping portion 48 includes an outward surface 50 and an inward surface 52. The inward surface 52 faces an inner surface of the trigger 18.

As shown in FIGS. 4B and 5B, the interior 56 of side 16 is shown to include protrusions 58 which mate with corresponding shaped recesses (not shown) within the interior of side 14 during manufacture. Alternatively, the protrusions 58 could be located on side 14 with recesses within interior 56, or some of the protrusions 58 could be located on both sides 14 and 16 with corresponding recesses oppositely positioned on sides 14 and 16. When assembled, an exterior 60 of the side 14 and the exterior 54 of side 16 preferably combine to form a smooth outer surface of the handle assembly 12 for gripping by the operator.

Turning now to FIGS. 6B and 7B, a cross-section of the sides 14 and 16 is shown. Specifically, a cross-section of side 16 taken along line 7-7 within FIG. 3B is shown in FIG. 7B. A cross-section of side 14 taken along the same location, that is, through the opening 44 for safety button 22, is shown in FIG. 6B. The opening 44 extends from the exterior 54 of side 16 to the exterior 60 of side 14. Thus, the safety button, as will be further described below, is accessible from either side 14 or 16 of the handle assembly 12.

Turning now to FIGS. 8B-11B, the trigger 18 is shown to include an inner surface 62 which, when assembled within the handle assembly 12, faces the inward surface 52 of the sides 14 and 16. Outer surface 64 is preferably smooth for grasping by an operator. The trigger 18 may include spring receiving member 66 for receiving a hook 21 (as shown in FIG. 1B) of the spring 20. As shown, the spring receiving member 66 is an opening, although other shapes, such as a hook shape would be within the scope of this invention. With the hook 21 of the spring 20 engaged within the spring receiving member 66 of the trigger 18 and a securing member 19 (FIG. 1B) of spring 20 securing the spring 20 to a protrusion 58 on side 16, the trigger 18 must expand the spring 20 when the inner surface 62 of the trigger 18 is compressed towards the inward surface 52 of the sides 14, 16. The spring 20 thus biases the trigger 18 in an “open” or “unsqueezed” configuration. That is, after the trigger 18 is squeezed, the spring 20 will return the trigger 18 to its original position when pressure on the trigger 18 is removed.

The trigger 18 further preferably includes a pivot rod 68 for pivotally securing the trigger 18 within the handle assembly 12, such as within opening 70 within interior 56 of side 16, as shown in FIG. 3B. Thus, when the trigger 18 is squeezed, it will pivot about the longitudinal axis of pivot rod 68 located within the sides 14, 16.

The trigger 18 further preferably includes a hook-shaped safety button engaging member 70 which includes an inner receiving pocket 72 which either hovers above or rests upon the safety button 22 or is received within one of the grooves of the safety button 22, as will be further described with reference to FIGS. 12B-13B.

Turning now to FIGS. 12B-13B, the safety button 22 preferably includes a pair of pins 74, 76. Each of the pins 74, 76 include an engageable end 78, 80, respectively, which protrude through the openings 44 of the sides 14, 16 and are accessible by the operator. Surrounding the pins 74, 76, respectively, are the springs 24, 26. The springs 24, 26 are seated within pockets 45 (FIGS. 6B and 7B) of the openings 44. The pockets 45 have a slightly larger diameter than the exteriormost openings 44 such that the springs 24, 26 received therein abut against a wall 43 within the pockets 45. The safety button 22 further preferably includes three ribs, shown collectively as ribs 82. The ribs 82 include a first side rib 84, a second side rib 86, and a middle rib 88. A first side gap 90 is created between the first side rib 84 and the middle rib 88 and a second side gap 92 is created between the second side rib 86 and the middle rib 88.

When assembled within the handle assembly 12, the safety button 22 is preferably centrally located, and spring biased to be centrally located, within the handle assembly 12 such that the safety button engaging member 70 of the trigger 18 abuts with the middle rib 88 when an attempt to squeeze the trigger 18 is made. However, when the engageable end 78 of the safety button 22 is depressed by an operator, the safety button 22 moves within the handle assembly 12 such that the safety button engaging member 70 of the trigger 18 will fall into the gap 90. Thus, the trigger 18 is now free to be squeezed by the operator. Likewise, when the engageable end 80 of the safety button 22 is depressed by an operator, the safety button 22 moves within the handle assembly 12 such that the safety button engaging member 70 of the trigger 18 will fall into the gap 92 freeing the trigger 18 to be moved by the operator. While the accessibility of the safety button 22 from either side 14 or 16 of the handle assembly 12 provides ease of use to the operator, it is within the scope of this invention to have the safety button accessible from only one side 14 or 16 of the handle assembly 12, which would thus require only one gap and only one pair of ribs in the safety button 22. Pressing the safety button 22 will preferably allow the safety button engaging member 70 to be retained between two adjacent ribs, i.e. ribs 84 and 88 or ribs 88 and 86, but with enough space within either gap 90 or 92 to allow movement of the safety button engaging member 70 during a squeeze of the trigger 18. The purpose of the safety button 22 is to prevent unintentional accidental firing of the device 10. Thus, preferably the safety button 22 is self-centering due to springs 24, 26. After depressing the safety button 22 and releasing the trigger 18 for movement, and after the trigger 18 is squeezed and released by the operator, the safety button 22 will preferably return to its center position, re-locking the trigger 18 from movement. Preferably, the device 10 is a one-time use instrument such that a ferrule 42 cannot be reloaded within the device.

Turning now to FIGS. 14B and 15B, and as additionally shown in FIG. 1B, the adjustment screw 28 is shown which facilitates ferrule loading by the manufacturer. The adjustment screw 28 preferably includes a distal end 94 and a proximal end 96. The distal end 94 may include a bulbous or larger diametered head 98. The proximal end 96 may include a slotted portion 100. A shaft 102 preferably connects the distal end 94 to the proximal end 96. Extending within the adjust screw 28 is a longitudinal bore 104 which extends along the longitudinal axis 106. The bore 104 may have a smaller inner diameter 103 within the head 98 than within the shaft 102. As demonstrated in FIG. 1B, the adjustment screw 28 is connected to a proximal end 32 of the central rod 30, which extends from a distal end 13 of the handle assembly 12. The adjustment screw 28 is preferably completely contained within the handle assembly 12 and is not accessible by the operator. During assembly, the adjustment screw 28 accepts the section 31, preferably threaded, of the proximal end 32 of the central rod 30 so that the length of the central rod 30 may be properly adjusted with respect to the tubular portion 38. The smaller inner diameter 103 is also preferably threaded such that the inner diameter 103 may be threaded to mate with threads on section 31 of rod 30. Turning the adjustment screw 28 after loading the ferrule 42 shortens the rod 30 and allows the distal end 108 of the hammer element 34 to retain the ferrule 42 in the ferrule accepting opening 142 in the tip 40. That is, the proper length of the central rod 30 with respect to the tubular portion 38 helps ensure that the ferrule 42 is retained within the distal end of the device 10. Also, the ability to correct the length of the central rod 30 using the adjustment screw 28 eliminates the need to require very tight tolerances during manufacture of the central rod 30, thus easing the manufacturing process of the device 10.

Turning now to FIGS. 16B-17B, the hammer element 34 is shown. The hammer element 34 includes a distal end 108 and a proximal end 110. The distal end 108 includes the hammer head 112. The hammer head 112 preferably includes a first camming surface 114 which engages with a camming surface on the tip 40 as will be further described. The first camming surface 114 is located on a first side 126 of the hammer element 34. The first camming surface 114 and the first side 126 form an obtuse angle as shown. Located on the second side 128 of the hammer element 34 is a ferrule engaging edge 116. An indent 118 may separate the ferrule engaging edge 116 and an edge 120. Alternatively edge 120 may be removed and replaced with a smooth continuous edge, continuous with second side 128. The hammer element 34 includes a central portion 122 of a selected width which is smaller in width than a proximal portion 124. The smaller width of the central portion 122 allows movement of the hammer head 112 within the tip 40. Each of the first side 126 and second side 128 may comprise a series of planar surfaces as shown in FIG. 17B, with planar sides 130 connecting the first and second sides 126, 128. Thus, the hammer element 34 preferably comprises a rectangular cross-section. As shown in FIG. 1B, the proximal end 110 of the hammer element 34 is mounted to the distal end 36 of the central rod 30.

Turning now to FIGS. 18B-21B, tip 40 is shown in detail. Tip 40 has a distal end 132 and a proximal end 134. The distal end 132 includes a hammer head receiving portion 136 which includes a second camming surface 138 (shown in FIG. 21B), which abuts with the first camming surface 114 of the hammer head 34 during retraction of the hammer element within the device 10. The second camming surface 138 forms part of a wall of the opening 140 of the tip 40. The opening 140 preferably extends the length of the tip 40 and has a rectangular cross section (as shown in FIG. 18B) throughout most of the tip 40 for receiving the rectangularly shaped hammer element 34. The distal end 132 of the tip 40 further includes a ferrule accepting opening 142 which shares open space with the opening 140. Thus, the opening at the distal end 132, as shown in FIGS. 19B and 20B is generally key-hole shaped. Proximal the ferrule accepting opening 142 is a cutting edge 144 formed on an inner wall of the tip 40 for cutting the suture material as will be further described below. Adjacent the cutting edge 144 is an aperture 146 within the tip 40. The aperture 146 allows the suture material to be threaded through the ferrule 42 from the distal end 132 and exit the aperture 146. The tip 40 preferably includes a proximal portion 148 having a reduced width. A wall 150 is formed between the proximal portion 148 and the hammer head receiving portion 136.

Turning now to FIG. 22B, the distal end 36 of central rod 30 is shown welded to the proximal end 110 of the hammer element 34 at area 152. Notch 154 is shown within the rod 30. Then, the tip 40 is installed upon the hammer element 34. Turning to FIG. 23B, the tubular portion 38 is shown welded or otherwise secured to the tip 40 at or about area 156. The tubular portion 38 overlaps the proximal portion 148 of the tip 40 and abuts the wall 150, so that preferably a smooth continual surface is provided between the tubular portion 38 and the tip 40. Notch 158 within tubular portion 38 coincides with notch 154 in the central rod 30. An anti-rotation feature is provided using the aligned notches 154, 158 during assembly by placing a pin, such as a square pin, into the notch area, thus preventing the rod 30 from rotating within the tubular portion 38. Longitudinal axis 106, which also runs through adjustment screw 28, extends generally through the central rod 30 and tubular portion 38.

FIGS. 24B and 25B show an exemplary ferrule 42 for use within the device 10, and more particularly for placement within the ferrule accepting opening 142 of the tip 40. The ferrule 42 includes a bore 168 which extends the length of the ferrule 42. Ferrule 42 preferably comprises an ovalized outer surface 164. The inner surface 166 of the ferrule 42 will contact the suture material upon compression, as will be described below. The ferrule 42 preferably comprises chamfered ends 160 and 162. The ends may be angled as shown by end 160 or more preferably rounded as shown by end 160. The ferrule 42 is preferably manufactured without burrs of any sort. The material selected for ferrule 42 is preferably annealed titanium, but may be formed from another deformable biocompatible material, such as another non-bioabsorbable material. Alternatively, the ferrule 42 may be formed from a bioabsorbable polymer.

FIGS. 26B-29B describe how the hammer element 34 and tip 40 cooperate to compress the ferrule 42, secure the suture threads therein, and cut the suture thread ends. The ferrule 42 is shown positioned within the ferrule accepting opening 142 and the suture threads 170 have been threaded through the ferrule 42 and exit the aperture 146 of the tip 40. FIG. 26B shows the hammer head 112 positioned within the tip 40 such that the first camming surface 114 abuts the second camming surface 138. In this initial position, the ferrule engaging edge 116 may abut the ferrule 42 and provide a slight compression of the ferrule 42 for retaining the ferrule 42 within the ferrule accepting opening 142. Turning now to FIG. 27B, as the hammer element 34 is drawn in the direction indicated by arrow 172, the hammer head 112 draws the first camming surface 114 along the second camming surface 138. In doing so, the hammer head 112 is brought closer to the ferrule 42 such that the ferrule engaging edge 116 begins to crimp or compress the ferrule 142. Turning to FIG. 28B, with the hammer element 34 continually drawn in the direction indicated by arrow 172, the first camming surface 114 is no longer in contact with the second canning surface 138, but the first side 126 abuts the inner surface of the opening 140 within tip 40 such that the ferrule engaging edge 116 continues to crimp the ferrule 42. As shown in FIG. 29B, after the hammer element 34 has been moved in the direction indicated by arrow 172 to completely crimp the ferrule 42, the ferrule engaging edge 116 moves towards the cutting edge 144 of the tip 40 until the suture threads 170 are trapped between the hammer head 112 and the cutting edge 144. A small amount of pressure from the hammer head 112 upon the cutting edge 144 will release the ends of the suture threads 170 as shown. Thus, the ferrule 42 is crimped and the suture ends are cut in one step. Additionally, the hammer element 34 does not contain the cutting edge, and therefore there is no risk of providing sharp edges to the ferrule 42 which will remain in the suture location.

FIGS. 30B-34B show one embodiment of a suture loading assembly 174 for assisting an operator in threading the suture threads 170 through the ferrule 42 after a suturing operation and before a suture securing operation. The suture loading assembly 174 preferably includes a body 176 from which extends a flexible loop 178, preferably made from suture material 186 or wire, such as stainless steel wire, and a cap 180. The body 176, as shown in FIG. 34B, includes a distal end 182 from which the loop 178 exits, and a proximal end 184. The body 176 further preferably includes a bore 188 (FIG. 31B) containing the suture material 186 from which the loop 178 is formed. As further shown in FIG. 31b, the body 176 further preferably comprises a step 190 for abutting with a stopping surface 192 within the cap 180. Additionally, the body 176 further includes an attaching member 194, which may have a clip-like shape as shown with a pair of legs 196, 198. The legs 196 and 198 preferably define a rounded receiving pocket 200 for receiving the tubular portion 38.

Because of the small size of the tubular portion 38, and thus the body 176, the suture loading assembly 174 further preferably includes the cap 180 for easy grasping and operating by an operator. The cap 180 includes openings 202 and 204 for receiving the body 176 and attaching member 194. The cap 180 further preferably includes sides 206 which have indents 208 for ease in grasping.

The suture loading assembly 174 is preferably pre-assembled upon the device 10 by the manufacturer. During assembly, the suture loading assembly 174 is preferably secured to the tubular portion 38 by inserting the tubular portion 38 into the opening 204 of the cap 180 and snapping the attaching member 194 onto the tubular portion 38. The loop 178 (which may be much longer than what is shown) may then be pushed into the opening 146 in the tip 40 and threaded through the ferrule 42 which is preloaded within the tip 40. Thus, a portion of the loop 178 will remain extended through the ferrule 42 and out the distal end 132 of the tip 40. Alternatively, the loop 178 could be threaded through the ferrule 42 in the manner described and then the suture loading assembly could be secured to the tubular portion 38.

When a suturing operation has been completed, and it is time to utilize the crimping and cutting device 10 for securing the suture, the ends of the suture material 170 may be simply threaded through the large opening provided in the loop 178. Then, the operator may grasp the cap 180, such as at indents 208, and then the operator may pull the suture loading assembly 174 in a proximal direction, towards the handle assembly 12. In doing so, the loop 178, which is flexible and collapsible, will pull the suture material 170 through the ferrule 42 and out the opening 146 in the tip 40. Because the suture material 170 is likely to be wet and slippery following the suture operation, the ability to thread the suture material 170 through the ferrule 42 using the suture loading assembly 174 eliminates any tedious operational steps.

Turning now to FIGS. 35B-38B, another embodiment of a suture loading assembly is shown. The suture loading assembly 250 is similar in use to the suture loading assembly 174, but embodies a slightly different design. The suture loading assembly 250 includes a wire loop 252 made of wire 253, a body 254, and a plug 256. During assembly, ends 258 of wire loop 252 may be trimmed at location 260 after installing plug 256 so that the wire loop 252 ends flush with a proximal end 262 of the body 254. The wire loop 252 preferably includes a tapered distal end 264, a widest portion 266, and a cross-over portion 268 where the wire 253 crosses over itself prior to running parallel into the body 254.

The body 254 includes a tapered nose section 270 having a distal end 272 and an opening 274. The opening 274 receives the wire 253 of the wire loop 252. As shown in FIG. 38B, the opening 274 leads to a longitudinal bore 276 having a main bore 280 with a first inner diameter, distal tapered section 278 having a smaller inner diameter than the first inner diameter, and a proximal bore 282 having a second inner diameter slightly larger than the first inner diameter, such that a stopping surface 284 is provided within the bore 276. During assembly, the plug 256 is inserted into the proximal end 262 of the body 254 for retaining the wire loop 252 within the body 254.

The body 254 further preferably includes an integral attaching member 286 which includes a pair of clip-like legs 288 separated by a slot 290 having a rounded end 292 for receiving the tubular portion 38. Each leg 288 further preferably includes an indented area 294 for ease in grasping.

As with the suture loading assembly 174, the suture loading assembly 250 is preferably pre-assembled upon the device 10 by the manufacturer. During assembly, the suture loading assembly 250 is preferably secured to the tubular portion 38 by inserting the tubular portion 38 into the slot 290 and snapping the attaching member 286 onto the tubular portion 38 with the tubular portion 38 residing in the rounded end 292 of the slot 290. The wire loop 252, which is sufficiently flexible, may be pushed into the opening 146 in the tip 40 and threaded through the ferrule 42 which is preloaded within the tip 40. Thus, a portion of the wire loop 252 will remain extended through the ferrule 42 and out the distal end 132 of the tip 40. Preferably, the wire loop 252 is preformed such that upon its exit through the ferrule 42, it will begin to open up automatically thus creating a stable opening, as opposed to suture material in which the opening may have to be created by separating the thread used in the loop 178 in the suture loading assembly 174. Alternatively, the wire loop 252 could be threaded through the ferrule 42 in the manner described and then the suture loading assembly 250 could be secured to the tubular portion 38. The ends of suture material 170 may be threaded through the large opening provided in the wire loop 252, and the operator may grasp the indented areas 294 and pull the suture loading assembly 252 in a proximal direction, towards the handle assembly, for pulling the suture material 170 through the ferrule 42 as previously described with the operation of the suture loading assembly 174.

FIGS. 39B and 40B show an alternate embodiment of a suture loading assembly 210, where halves of the suture loading assembly 210 are depicted in FIGS. 39B-40B. Looking at the suture loading assembly from a distal location, i.e. from the funnel 212, FIG. 40B shows a left half 214 and FIG. 39B depicts a right half 216. The two halves 214, 216 preferably snap onto the distal end of the device 10 for threading of the ferrule 42. The suture material 170 may be threaded through a funnel 212 created by a joining of the two halves 214, 216. Then, the threads 170 would go directly into the ferrule 42 after being pushed into the funnel 212. After the ferrule 42 is threaded, the wings 218, 220 may be squeezed together to release the suture loading assembly 210 from the distal end of the device 10. Thus, with the suture material 170 threaded through the ferrule 42, the device 10 may be inserted near the suture location (the area where the body was closed by the suture material 170) to secure the ferrule 42 upon the suture material 170.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A combination wound suturing and crimping and cutting device, comprising:
a suturing device portion, the suturing device portion including a tissue receiving gap at a distal portion and a needle configured to traverse the tissue receiving gap to effectuate placement of suture therethrough; and
a crimping and cutting device portion, the crimping and cutting device portion including a ferrule configured to receive suture through a portion thereof, the ferrule housed in a distal tip portion, and crimping and cutting surfaces that are configured to crimp the ferrule, thereby securing the suture received therein, and to cut the suture trailing proximally from the ferrule.

2. The combination wound suturing and crimping and cutting device of claim 1, wherein the crimping and cutting device portion is configured to detach from the suturing device portion.

3. The combination wound suturing and crimping and cutting device of claim 1, wherein the crimping and cutting portion is configured to nest within a portion of the suturing device portion.

4. The combination wound suturing and crimping and cutting device of claim 1, wherein the crimping and cutting portion further includes a suture loading device, the suture loading device configured to pull a portion of suture from the body of the suturing portion and to load the suture through the ferrule housed in the crimping and cutting portion.

5. The combination wound suturing and crimping and cutting device of claim 1, wherein the wound suturing portion comprises a housing and an elongated shaft connected thereto and at least one needle within the shaft, the at least one needle configured to travel distally across a tissue engaging gap within a tissue engaging section positioned distally from the housing on said shaft, wherein the tissue receiving gap has two opposing surfaces into which one side of a wound can be received, wherein the gap is shaped to have a depth to facilitate the placement of the edge of a wound therein such that at least one surface comprises a stop surface, and wherein the stop surface is squared at a middle portion thereof to provide good tactile feel to the surgeon when tissue is engaged.

6. The combination wound suturing and crimping and cutting device of claim 1, wherein, wherein the crimping and cutting portion comprises a hammer head having a first side and an opposite second side, a ferrule engaging edge located on the second side, a tip having a distal end and a proximal end, the tip having a hammer head opening for receiving the hammer head, the hammer head opening extending from the distal end to the proximal end, the tip further having a ferrule accepting opening near the distal end, and a cutting edge within the hammer head opening of the tip, the cutting edge located proximally of the ferrule accepting opening.
